(19) United States
(12) Patent Application Publication
(10) Pub. No.: US 2026/0157965 A1
(43) Pub. Date: Jun. 11, 2026

(54) GOLD NANOPARTICLE COMPOSITION AND USES THEREOF

(71) Applicant: NATIONAL CHENG KUNG UNIVERSITY, Tainan City (TW)

(72) Inventors: Chen-Sheng YEH, Tainan City (TW); Ying-Chi CHEN, Tainan City (TW)

(21) Appl. No.: 18/971,436

(22) Filed: Dec. 6, 2024

(51) Int. Cl. A61K 9/127; A61K 9/14; A61K 33/242; A61K 47/42; A61P 35/00

(52) U.S. Cl. CPC A61K 9/127; A61K 9/14; A61K 33/242; A61K 47/42; A61P 35/00

(57) ABSTRACT

The present disclosure relates to a gold nanoparticle composition. The gold nanoparticle composition comprising a gold nanoparticle, a liposome encapsulating the gold nanoparticle, and a heme integrated into the liposome. A method for treating cancer and a method for inducing lipid oxidation, membrane potential change and/or ROS generation are also provided.

### FIELD OF THE INVENTION

The present disclosure relates to a field of nanoparticles. Particularly, the present disclosure pertains to a gold nanoparticle composition and uses thereof.

### BACKGROUND OF THE INVENTION

Gold nanoparticles are a type of nanomaterial that contain small particles of gold, typically ranging in size from 1 to 500 nanometers. These nanoparticles have unique physical, chemical, and optical properties that make them valuable for a wide range of applications in fields such as medicine, electronics, and catalysis.

In medicine, gold nanoparticles are being explored for use in targeted drug delivery, cancer therapy, and diagnostic imaging.

Gold nanoparticles are a versatile and promising nanomaterial with a wide range of potential applications.

### SUMMARY OF THE INVENTION

The present disclosure is at least based on providing novel gold nanoparticles and their clinical applications.

The present disclosure provides a gold nanoparticle composition comprising:
a gold nanoparticle;
a liposome encapsulating the gold nanoparticle; and
a heme integrated into the liposome.

In some embodiments, the gold nanoparticle ranges from 1 nm to 500 nm, from 10 nm to 450 nm, from 20 nm to 400 nm, from 30 nm to 350 nm, from 40 nm to 300 nm, from 50 nm to 250 nm, from 60 nm to 200 nm, from 70 nm to 190 nm, from 80 nm to 180 nm, from 90 nm to 170 nm, from 100 nm to 160 nm, from 110 nm to 150 nm, from 100 nm to 140 nm, from 110 nm to 130 nm, or from 115 nm to 125 nm, in diameter.

In some embodiments, the gold nanoparticle is conductive.

In some embodiments, a surface of the gold nanoparticle is modified. In some further embodiments, a surface of the gold nanoparticle is modified with PEGylation.

In some embodiments, the heme is contained in a cytochrome.

In some embodiments, the cytochrome contains a plurality of hemes, for example, the cytochrome contains two, three, four, five, six, seven, eight, nine, ten, eleven, twelve, thirteen, fourteen, or fifteen hemes. In some embodiments, the cytochrome is MtrA, MtrB, MtrC, OmcA or OmcB.

In some embodiments, the heme comprises Fe. In some embodiments, a molecular ratio of Fe to the heme ranges from 1:1 to 1:4 or from 1:2 to 1:3.

In some embodiments, a molecular ratio of the gold nanoparticle to the liposome ranges from 1 to 15, from 1 to 14, from 1 to 13, from 1 to 12, from 1 to 11, from 1 to 10, from 1 to 9, from 1 to 8, from 1 to 7, from 1 to 6, from 1 to 5, from 1 to 4, from 1 to 3, from 1 to 2, or from 1 to 1.

In some embodiments, a molecular ratio of the cytochrome to the gold nanoparticle ranges from 1 to 6000, from 1 to 5500, from 1 to 5000, from 1 to 4500, from 1 to 4000, from 1 to 3500, from 1 to 3000, from 1 to 2500, from 1 to 2000, from 1 to 1800, from 1 to 1600, from 1 to 1400, from 1 to 1200, from 1 to 1000, from 1 to 900, from 1 to 800, from 1 to 700, from 1 to 600, from 1 to 500, from 1 to 400, from 1 to 300, from 1 to 200, from 1 to 100, from 1 to 80, from 1 to 60, from 1 to 50, from 1 to 40, from 1 to 30, from 1 to 20, from 1 to 10, from 1 to 9, from 1 to 8, from 1 to 7, from 1 to 6, from 1 to 5, from 1 to 4, from 1 to 3, from 1 to 2, 1, from 1 to 890, from 1 to 880, from 1 to 870, from 1 to 860, from 1 to 850, from 1 to 840, from 1 to 830, from 1 to 820, or from 1 to 810.

In some embodiments, the liposome and the heme have a redox potential ranging from −0.4 eV to +0.3 eV, from −0.35 eV to +0.25 eV, from −0.3 eV to +0.2 eV, from −0.25 eV to +0.15 eV, from −0.2 eV to +0.1 eV, from −0.15 eV to +0.05 eV, from —0.1 eV to 0 eV, from −0.05 eV to 0 eV.

In some embodiments, the liposome and the heme are provided by Shewanella oneidensis MR-1.

The present disclosure provides a pharmaceutical composition comprising the gold nanoparticle composition as disclosed herein and optionally a pharmaceutically acceptable carrier.

The present disclosure provides a method for treating cancer comprising administering the gold nanoparticle composition as disclosed herein to a subject in need. Alternatively, the present disclosure provides use of the gold nanoparticle composition as disclosed herein in the manufacture of a medicament for treating cancer in a subject in need.

In some embodiments, the cancer is a solid cancer. Examples of the cancer include, but are not limited to, squamous cell cancer, lung cancer, cancer of the peritoneum, hepatocellular cancer, gastric or stomach cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, lymphomas, leukemias, myelomas and myeloproliferative neoplasms.

In some embodiments, the cancer is liver cancer, lung cancer, pancreatic cancer, breast cancer, cervical cancer, or colorectal cancer.

The present disclosure provides a method for inducing lipid oxidation, membrane potential change and/or reactive oxygen species (ROS) generation comprising administering the gold nanoparticle composition of as disclosed herein to a subject in need. Alternatively, the present disclosure provides use of the gold nanoparticle composition as disclosed herein in the manufacture of a medicament for inducing lipid oxidation, membrane potential change and/or reactive oxygen species generation in a subject in need.

In some embodiments, the lipid oxidation or membrane potential change occurs in mitochondrial or endoplasmic reticulum.

### BRIEF DESCRIPTION OF DRAWINGS

FIGS. 1A to 1F show characterization of Au@MIL NPs. FIG. 1A shows that high-resolution TEM images of Au NPs, MILs, and Au@MIL NPs (with the red arrow indicating the MIL membrane area) were captured. The lattice spacing of the (200) plane is shown in Au@MIL NPs, and the electron diffraction pattern confirms a crystalline structure of Au@MIL. FIG. 1B shows that energy-dispersive X-ray spectroscopy (EDS) line scanning and FIG. 1C shows EDS mapping along the Au@MIL NPs reveal P κα1 signal from the phosphorus element in MIL distributed on Au@MIL NPs. FIG. 1D shows that the Fourier-transform infrared (FTIR) spectra of Au NPs, MILs, and Au@MIL NPs were analyzed. FIG. 1E shows that SDS-PAGE results of S. oneidensis MR-1, MILs, Au@MIL NPs, Au NPs, and Au@PEG NPs display protein bands stained with Coomassie Brilliant Blue (CBB) for total protein detection and TMBZ for haem-based protein detection. FIG. 1F shows that the fluorescence spectra of the membrane-specific dye FM 4-64 with maximum fluorescence at 680 nm confirm the presence of the lipid membrane on the surface of the Au NPs.

FIG. 2 shows Cytotoxicity and colony formation assays of 70 nm Au-based NPs. Colony assay of 70 nm Au-based NPs in NeHepLxHT, M10, Hep G2, HA22T, and MDA-MB-231 cells (n=3). Cells were incubated with 300 ppm of Au-based NPs for 72 hours and maintained without them for another 14 days. The formation of clones was fixed and stained with 0.5% crystal violet, and the results were measured using ImageJ™ software. Error bars represent standard deviation, and p-values were calculated using one-way ANOVA.

FIGS. 3A and 3B show size-dependent cytotoxicity and colony formation assays of Au@MIL NPs. FIG. 3A shows that the size and incubation-dependent curve of Au@MIL NPs illustrates the impact of NP size on enhancing the cytotoxic effect against Hep G2 cells over 24, 48, and 72-h intervals. FIG. 3B shows colony assay of 100 nm Au@MIL in NeHepLxHT, M10, Hep G2, HA22T, and MDA-MB-231 cells (n=3). Cells were incubated with 300 ppm of Au@MIL for 72 hours and maintained without them for another 14 days. The formation of clones was fixed and stained with 0.5% crystal violet, and the results were measured using ImageJ™ software. The error bars represent the standard deviation, and the p-values were calculated using the student's t-test.

FIGS. 4A to 4F show mitochondrial functions and oxidative stress responses in cancerous cells treated with Au@MIL NPs. FIGS. 4A and 4B show analysis of mitochondrial membrane potential in Hep G2 and MDA-MB-231 cells. Cells were treated with 300 ppm of 100 nm Au@MIL for 24, 48, and 72 hours, then assessed using the JC- 1 Mitochondrial Membrane Potential Assay Kit. The stained cells were observed with a CKX53 fluorescence microscope and analyzed using ImageJ™ software. Corresponding quantification of relative fluorescence signal is also provided. The error bars represent standard deviation and p-values calculated by Student's t-test. FIG. 4C shows Fluorescence imaging of mitochondrial ATP fluctuations with 100 nm Au@MIL NPs. To detect mitochondrial ATP production, cells were incubated with the fluorescent probe ATP Red-1 at 37° C. for 15 minutes. Images were captured using a 20X objective lens on a fluorescence microscope. These images are representative of three independent experiments. Fluorescence intensity was measured using ImageJ™ software. Data are presented as mean ±s.e.m., and statistical analysis was conducted using a two-tailed Student's t-test. FIGS. 4D and 4E show that oxygen consumption rate (OCR) analysis in Hep G2 cells was performed using the Seahorse XFe24 Extracellular Flux Analyzer. Cells were treated with 300 ppm of 100 nm Au@MIL for 24 hours, then the XF Cell Mito Stress Test was conducted following treatments with oligomycin (2 μM), FCCP (2 μM), and a combination of rotenone (2 μM) and antimycin A (2 μM). Basal respiration, ATP production, and maximal respiration capacities were measured based on OCR values at different experimental time points. The error bars represent standard deviation and p-values calculated by Student's t-test. FIG. 4F shows analysis of lipid peroxidation induced by 100 nm Au@MIL NPs using C 11-BODIPY 581/591 staining. Representative images of fluorescence signal showed lipid peroxidation in Hep G2 cells treated with Au@MIL NPs at a concentration of 300 ppm for 24 and 48 hours. Ferroptocide, was used as a positive control to induce rapid ferroptotic death by incubating Hep G2 cells with 50 μM Ferroptocide for 1 hour. Cells were stained with 4 μg/mL Hoechst 33258 (blue) to label nuclei and the C11-BODIPY 581/591 probe (10 μM for 1 hour) to label lipid peroxidation. Confocal imaging was then used to analyze the stained cells. These images are representative of three independent experiments. Scale bars are 20 μm. Corresponding quantification of relative lipid peroxidation amounts is also provided.

FIGS. 5A to 5D shows In vivo anti-tumor efficacy of Au@MIL in orthotopic HepG2-Red-FLuc HCC xenograft mice. HepG2-Red-FLuc orthotopically injected xenograft mice (8-10 weeks, male) were administered with a single dose of sterilized PBS (100 μL), 3000 ppm of 100 nm Au@PEG (in 100 μL sterilized PBS), and 3000 ppm of 100 nm Au@MIL (in 100 μL sterilized PBS) via intravenous injection. FIG. 5A shows that ICP-MS assessed the biodistribution of Au@MIL at different post-treatment time points (1, 4, 8, 24, and 72 hours) in the following samples: heart, lung, spleen, kidney, liver, HCC tumor, and feces collected from treated mice (n=3). FIG. 5B shows orthotopic tumor growth of HepG2-Red-FLuc cells in xenograft mice treated with PBS, Au@PEG, and Au@MIL monitored by IVIS system (n=5). The luminance signal was measured as the total flux ratio on post-treatment Day 0, 4, 7, 11, and 14 compared to Day 0. The error bars represent standard deviation and p-values calculated by One-way ANOVA. FIG. 5C shows that ex vivo luminance signals (total flux) of HepG2-Red-FLuc cells in the liver, collected from mice treated with PBS, Au@PEG, and Au@MIL after 14 days, were monitored using the IVIS system (n=5). The error bars represent standard deviation and p-values calculated by One-way ANOVA. FIG. 5D shows that the survival rate of HepG2-Red-FLuc HCC xenograft mice treated with PBS, Au@PEG, and Au@MIL (n=5) was assessed, and p-values were calculated using the log-rank test.

FIGS. 6A to 6E show comprehensive evaluation of Au@MIL NPs: cellular uptake, electron transfer, spectral analysis, and cytotoxicity under hypoxic conditions. FIG. 6A shows that the cellular uptake of Au@MIL NPs was assessed in two normal cell lines (NeHepLxHT, M10) and three cancer cell lines (Hep G2, HA22T, and MDA-MB-231) treated with 100 nm Au@MIL NPs at a concentration of 300 ppm for 48 hours. The data are presented as mean ±s.e.m. FIG. 6B shows a schematic illustration depicting the transfer of electrons to Au NPs through the MIL. The redox potentials of Au NPs and MIL versus NHE are also indicated. FIG. 6C shows that the UV-visible spectra of Au NPs, Au@MIL NPs, and Au@MIL NPs after 72 hours of incubation with cells were analyzed. The corresponding SPR and full width at half maximum (FWHM) bandwidth values are provided in the table below. FIG. 6D shows that the XANES spectra at the Au L3-edge were obtained for Au NPs, Au@MIL NPs, Au@MIL NPs after 72 hours of cell incubation, and Au foil. FIG. 6E shows that TEM images of 100 nm Au@SiO₂ NPs and Au@SiO₂@MIL NPs were captured. The zoomed-in images reveal that the dark spheres represent Au NPs, the gray layer corresponds to SiO₂, and the transparent layer on the outermost surface is MIL.

### DETAILED DESCRIPTION OF THE INVENTION

It is to be understood that this invention is not limited to the particular materials and methods described herein. It is also to be understood that the terminology used herein is for the purpose of describing particular embodiments and is not intended to limit the scope of the present invention, which will be limited only by the appended claims.

It must be noted that, as used in this specification and the appended claims, the singular forms “a,” “an,” and “the” include plural referents unless the content clearly dictates otherwise.

As used herein, the terms “comprises,” “comprising,” “includes,” “including,” “has,” “having, “contains,” “containing,” or any other variation thereof, are intended to cover a non-exclusive inclusion. A composition, mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive “or” and not to an exclusive “or.”

The term “gold nanoparticle” as referred to herein means a particle including gold metal framework having at least one special dimension measurable less than a micron in length. Nanoparticles include conventionally known nanoparticles such as nanorods, nanospheres and nanoplatelets. In various embodiments, for example, nanospheres can be a rod, sphere, or any other three dimensional shape. In some embodiments, the gold nanoparticle ranges from 1 nm to 500 nm in diameter. Those of skill in the art will understand that the size of the gold nanoparticle can be designed to have specific properties for different applications.

As used herein, a liposome is self-assembling, substantially spherical vesicle comprising a lipid bilayer that encircles a core, which can be aqueous, wherein the lipid bilayer comprises amphipathic lipids having hydrophilic headgroups and hydrophobic tails, in which the hydrophilic headgroups of the amphipathic lipid molecules are oriented toward the core or surrounding solution, while the hydrophobic tails orient toward the interior of the bilayer. The lipid bilayer structure thereby comprises two opposing monolayers that are referred to as the “inner leaflet” and the “outer leaflet,” wherein the hydrophobic tails are shielded from contact with the surrounding medium. The “inner leaflet” is the monolayer wherein the hydrophilic head groups are oriented toward the core of the liposome. The “outer leaflet” is the monolayer comprising amphipathic lipids, wherein the hydrophilic head groups are oriented towards the outer surface of the liposome. The term “liposome” encompasses both multilamellar liposomes comprised of anywhere from two to hundreds of concentric lipid bilayers alternating with layers of an aqueous phase and unilamellar vesicles that are comprised of a single lipid bilayer. Methods for making liposomes are well known in the art and are described elsewhere herein.

As used herein, a “composition” refers to any mixture of two or more products. It can be a solution, a suspension, liquid, powder, a paste, aqueous or non-aqueous formulations or any combination thereof. As used herein, the term “pharmaceutical composition” refers to a chemical compound or composition capable of inducing a desired therapeutic effect in a subject. In certain embodiments, a pharmaceutical composition includes an active agent (such as gold nanoparticle, liposome and heme), which is the agent that induces the desired therapeutic effect. In certain embodiments, a pharmaceutical composition includes inactive ingredients such as carriers and excipients.

The term “pharmaceutically acceptable” as used herein, refers to agents that, within the scope of sound medical judgment, are suitable for use in contact with tissues of human beings and/or animals without excessive toxicity, irritation, allergic response, or other problem or complication, commensurate with a reasonable benefit/risk ratio.

As used herein, “effective amount” means an amount of an agent to be delivered (e.g., drug, therapeutic agent, diagnostic agent, prophylactic agent, etc.) that is sufficient, when administered to a subject suffering from or susceptible to a disease, disorder, and/or condition, to treat, improve symptoms of, diagnose, prevent, and/or delay the onset of the disease, disorder, and/or condition.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the terms “treatment,” “treating,” and the like, cover any treatment of a disease in a mammal, particularly in a human, and include: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The present disclosure provides a newly redox balance therapeutic strategy to disrupt redox equilibrium using gold nanoparticles (Au NPs) as electron sinkers combined with electroactive membranes, such as liposomes.

In one embodiment, the present disclosure provides a gold nanoparticle composition comprising:
a gold nanoparticle;
a liposome encapsulating the gold nanoparticle; and
a heme integrated into the liposome.

Using common gold NPs as electron sinkers, combined with electroactive membrane, enables the autonomous transfer of electrons from cancer cells to Au NPs, thereby disrupting the redox processes within cancer cells and causing their death. This simple design and strategy can be applied experimentally to induce the death of various types of cancer cells.

In one embodiment, the gold nanoparticle ranges from 1 nm to 500 nm in diameter. Without being limited by theory, it is believed that the size of the gold nanoparticle affects the efficacy of the composition, and a larger size attracts more electrons.

In some embodiments, the gold nanoparticle is conductive. In some embodiments of the disclosure, the gold nanoparticle is conductive. In some embodiments, the gold nanoparticle may include a core of atoms that form inorganic conductors. In some embodiments, the atoms form oxides, nitrides, and sulfides. In some embodiments, suitable materials for fabrication of the gold nanoparticles further comprise Ag, Cu, Li, Pt, alumina, titania, magnetite, or FePt.

In some embodiments, the gold nanoparticle further comprise a second nanoparticle. In some embodiments, the second nanoparticle is a quantum dot, a magnetic nanoparticle, an oxide nanoparticle, or a nanoparticle carrying negatively charged surface groups (carboxylates, borates, etc.). Specific examples of such quantum dots include, but are not limited to, those comprising materials including CdS, CdSe, CdTe, ZnS, ZnSe, ZnTe, GaN, GaP, GaAs, GaSb, AlN, AlP, AlAs, AlSb, InP, InAs, and InSb. Where quantum dots having a core-shell structure are used, the core may be overcoated with a material selected from the group consisting of ZnS, ZnSe, ZnTe, CdS, CdSe, CdTe, HgS, HgSe, HgTe, AlN, AlP, AlAs, AlSb, GaN, GaP, GaAs, GaSb, GaSe, InN, InP, InAs, InSb, TlN, TlP, TlAs, TlSb, PbS, PbSe, PbTe, Au, Ag, Cu, Co, Ni, Pt, Pd, and mixtures thereof.

In some embodiments, a surface of the gold nanoparticle is modified. For stable gold nanoparticle, one or multiple different functional ligands directly bound to the surfaces is fabricated. Also, the surface coverage amount of functional ligands on the surface of the gold nanoparticle can be tuned to be any percent value between 0 and 100%. All of these unique properties make the gold nanoparticle stable in the liquid, and with less or no need for stabilizing agents.

Among the molecules used for surface functionalization of the gold nanoparticle, polyethyleneglycol (PEG), or more specifically thiolated polyethyleneglycol (SH-PEG), is one of the more important and widely used species. Many other ligands can be used to functionalize the gold nanoparticle including aptamers, generally through binding at a thiol functionality linked to the aptamer. The surface modification of gold nanoparticles with PEG is often referred to as “PEGylation.” Since the layer of PEG on the surface of gold nanoparticles can help to stabilize the gold nanoparticles in an aqueous environment by providing a stearic barrier between interacting gold nanoparticles, PEGylated gold nanoparticles are much more stable at high salt concentrations.

According to the present disclosure, the liposome is used for encapsulating the gold nanoparticle; and carrying the heme which is integrated into the liposome as a membrane-integrated liposome (MIL). Without being limited by theory, it is believed that the membrane-integrated liposome, which contains heme, has the ability to transfer electrons. The heme promotes the transfer of electrons to the interior of the gold nanoparticle, which in turn enhances the therapeutic effect.

In some embodiments, the heme is contained in a protein forming a heme protein. The term “heme protein” is used herein to include any member of a group of proteins containing heme as a prosthetic group. Non-limiting examples of heme proteins include globins, cytochromes, oxidoreductases, any other protein containing a heme as a prosthetic group, and combinations thereof. Heme-containing globins include, but are not limited to, hemoglobin, myoglobin, and combinations thereof. Heme-containing cytochromes include, but are not limited to, cytochrome P450, cytochrome b, cytochrome c1, cytochrome c, and combinations thereof. Heme-containing oxidoreductases include, but are not limited to, a catalase, an oxidase, an oxygenase, a haloperoxidase, a peroxidase, and combinations thereof.

In certain instances, the heme proteins are metal-substituted heme enzymes containing protoporphyrin IX or other porphyrin molecules containing metals other than iron, including, but not limited to, cobalt, rhodium, copper, ruthenium, and manganese, which are active cyclopropanation catalysts.

In some embodiments, the heme protein is a member of one of the enzyme classes such as L-lactate dehydrogenase, polyvinyl alcohol dehydrogenase (cytochrome), methanol dehydrogenase (cytochrome c), alcohol dehydrogenase (quinone), formate dehydrogenase-N, alcohol dehydrogenase (azurin), gluconate 2-dehydrogenase (acceptor), fructose 5-dehydrogenase, cellobiose dehydrogenase (acceptor), alkan-1-ol dehydrogenase (acceptor), glutamyl-tRNA reductase, indole-3-acetaldehyde oxidase, aldehyde dehydrogenase (pyrroloquinoline-quinone), fumarate reductase (NADH), succinate dehydrogenase (ubiquinone), fumarate reductase (menaquinone), succinate dehydrogenase, methylamine dehydrogenase (amicyanin), aralkylamine dehydrogenase (azurin), methylenetetrahydrofolate reductase [NAD(P)H], spermidine dehydrogenase, NAD(P)H oxidase, nitrate reductase (NADH), nitrate reductase [NAD(P)H], nitrate reductase (NADPH), nitric oxide reductase [NAD(P), nitrous oxide-forming], nitrite reductase (NO-forming), nitrite reductase (cytochrome; ammonia-forming), trimethylamine-N-oxide reductase (cytochrome c), nitric oxide reductase (cytochrome c), hydroxylamine dehydrogenase, hydroxylamine oxidase (cytochrome), nitrate reductase (quinone), nitric oxide reductase (menaquinol), nitrite dismutase, ferredoxin-nitrite reductase, fenedoxin-nitrate reductase, nitrate reductase, hydrazine oxidoreductase, sulfite reductase (NADPH), sulfite dehydrogenase, thiosulfate dehydrogenase, sulfide-cytochrome-c reductase (flavocytochrome c), dimethyl sulfide: cytochrome c2 reductase, sulfite oxidase, sulfite reductase (ferredoxin), CoB-CoM heterodisulfide reductase, sulfite reductase, adenylyl-sulfate reductase, hydrogensulfite reductase, cytochrome-c oxidase, nitrate reductase (cytochrome), ubiquinol-cytochrome-c reductase, catechol oxidase, caldariellaquinol oxidase (H+-transporting), L-ascorbate oxidase, photosystem II, ubiquinol oxidase (H+-transporting), ubiquinol oxidase, menaquinol oxidase (H+-transporting), plastoquinol-plastocyanin reductase, cytochrome-c peroxidase, catalase, peroxidase, chloride peroxidase (vanadium-containing), bromide peroxidase (heme-containing), iodide peroxidase, chloride peroxidase, L-ascorbate peroxidase, manganese peroxidase, lignin peroxidase, versatile peroxidase, dye decolorizing peroxidase, catalase-peroxidase, unspecific peroxygenase, myeloperoxidase, plant seed peroxygenase, fatty-acid peroxygenase, cytochrome-c3 hydrogenase, hydrogen: quinone oxidoreductase, hydrogenase (acceptor), 2,5-dihydroxypyridine 5,6-dioxygenase, tryptophan 2,3-dioxygenase, chlorite O₂-lyase, acetylacetone-cleaving enzyme, indoleamine 2,3-dioxygenase, linoleate 8R-lipoxygenase, tryptophan 2′-dioxygenase, flavanone 3-dioxygenase, nitric oxide dioxygenase, nitric-oxide synthase (NADPH dependent), cholesterol 7alpha-monooxygenase, tyrosine N-monooxygenase, sterol 14 alpha-demethylase, N-methylcoclaurine 3′-monooxygenase, magnesium-protoporphyrin IX monomethyl ester, (oxidative) cyclase, 2-hydroxyisoflavanone synthase, cholesterol 24-hydroxylase, 5-epiaristolochene 1,3-dihydroxylase, vitamin D3 24-hydroxylase, beta-carotene 3-hydroxylase, cholest- 4-en-3-one 26-monooxygenase, 3-ketosteroid 9alpha-monooxygenase, linalool 8-monooxygenase, 1,8-cineole 2-endo-monooxygenase, vitamin D 25-hydroxylase, unspecific monooxygenase, camphor 5-monooxygenase, cholesterol monooxygenase (side-chain-cleaving), steroid 15beta-monooxygenase, spheroidene monooxygenase, tyrosinase, stearoyl-CoA 9-desaturase, linoleoyl-CoA desaturase, biflaviolin synthase, prostaglandin-endoperoxide synthase, heme oxygenase, steroid 17alpha-monooxygenase, steroid 21-monooxygenase, 4-methoxybenzoate monooxygenase (O-demethylating), carotene epsilon-monooxygenase, ascorbate ferrireductase (transmembrane), iron: rusticyanin reductase, xanthine dehydrogenase, lupanine 17-hydroxylase (cytochrome c), 4-methylphenol dehydrogenase (hydroxylating), ethylbenzene hydroxylase, chlorate reductase, selenate reductase, diguanylate cyclase, histidine kinase, cyclic-guanylate-specific phosphodiesterase, colneleic acid/etheroleic acid synthase, cystathionine beta-synthase, hydroperoxide dehydratase, colneleate synthase, chromopyrrolate synthase, guanylate cyclase, sirohydrochlorin cobaltochelatase, aliphatic aldoxime dehydratase, phenylacetaldoxime dehydratase, prostaglandin-E synthase, prostaglandin-I synthase, thromboxane-A synthase, 9,12-octadecadienoate 8-hydroperoxide 8R-isomerase, 9,12-octadecadienoate 8-hydroperoxide 8 S-isomerase, or cobaltochelatase. In other embodiments, the heme enzyme is a variant or homolog of a member of one of the enzyme classes. In yet other embodiments, the heme enzyme comprises or consists of the heme domain of a member of one of the enzyme classes or a fragment thereof (e.g., a truncated heme domain).

In some embodiments, the heme is contained in a cytochrome. In some embodiments, the cytochrome contains a plurality of hemes, such as ten hemes. In some embodiments, the cytochrome is MtrA, MtrB, MtrC, OmcA or OmcB.

In some embodiments, the heme comprises Fe. In some embodiments, a molecular ratio of Fe to the heme ranges from 1:1 to 1:4 or from 1:2 to 1:3.

A molecular ratio of the gold nanoparticle to the liposome may be varied. In some embodiments, a molecular ratio of the gold nanoparticle to the liposome ranges from 1 to 15. In some embodiments, a molecular ratio of the gold nanoparticle to the liposome is 10, exhibiting optimized performance of the electronic transfer and the overall system and showing the most significant results and stability.

In another aspect, a molecular ratio of the cytochrome to the gold nanoparticle may be varied. Considering that the amount of cytochrome carried by each gold nanoparticle (Au NP) varies depending on the size of the gold nanoparticle, the range of the molecular ratio of the cytochrome to the gold nanoparticle ranges from 1 to 6000. In some further embodiments, a molecular ratio of the cytochrome to the gold nanoparticle ranges from 1 to 890, from 1 to 880, from 1 to 870, from 1 to 860, from 1 to 850, from 1 to 840, from 1 to 830, from 1 to 820, or from 1 to 810. This range allows the amount of cytochrome to be adjusted with the size and surface area of the nanoparticles for optimal electron transfer and biocompatibility.

In some embodiments, the liposome and the heme have a redox potential ranging from −0.4 eV to +0.3 eV depending on the type of cytochrome and its environment.

In some embodiments, the liposome and the heme are provided by Shewanella oneidensis MR-1. Shewanella oneidensis MR-1, renowned for its remarkable extracellular electron transfer prowess, efficiently channels metabolic electrons to external acceptors. The outer membrane of this microorganism hosts pivotal proteins like MtrA, MtrB, and MtrC, instrumental in ferrying electrons to its surface. MtrC and OmcA proteins on the bacterial surface further aid in electron transfer to surrounding metal ions. An innovative method, liposome fusion-induced membrane exchange (LIME), allows for the extraction of electroactive membranes from these bacteria, yielding membrane-integrated liposomes (MIL) abundant in c-type cytochromes but free of intact bacteria (Chen, Y. C. et al. Nat. Nanotechnol. 18, 1492-1501 (2023)). These MIL structures, when coupled with TiO₂ NPs, generate electrons upon X-ray exposure, initiating superoxide production via the electroactive membrane. This underscores the vital role of membranes in triggering outward electron transport, underscoring their significance in orchestrating electron transport mechanisms and signaling their importance in cellular electron dynamics. Moreover, these electroactive membranes exhibit bidirectional electron transfer capabilities. The interplay between inward and outward electron movements across biological membranes adds complexity to electron transfer processes, showcasing the versatility of membranes in cellular electron transport.

In some embodiments, in the electroactive membrane of Shewanella oneidensis MR-1, cytochrome, a protein with a redox potential of −0.3 eV, plays a pivotal role. This potential is more negative than the Fermi level of Au, typically ranging from +0.35 to +0.45 eV compared to the Normal Hydrogen Electrode (NHE). Consequently, facilitated by cytochrome, we propose that extracellular electrons can potentially flow to Au, indicating Au's potential as an electron sink within specific electron transfer pathways. Ten heme groups construct an electron channel within cytochromes that allow efficient electron hopping and transferring through the redox cycle of Fe²⁺and Fe³⁺between these hemes, thus endowing an idiographic electroactive feature. Building upon this understanding, here we present a newly cancer redox balance therapeutic strategy revolving around Au@MIL NPs. Our goal is to disrupt the redox equilibrium within cancer cells, capitalizing on their highly active redox systems. By utilizing Au@MIL NPs, we aim to redirect electron transfer pathways within these cancer cells (i.e. directing electron transfer from cancer cells to Au NPs), disrupting their redox balance and undermining their survival mechanisms. Notably, a size effect is observed in Au NPs in this study, with larger size demonstrating greater efficacy in eradicating cancer cells. Significant observation shows that Au@MIL effectively induces cellular damage and death in hypoxic condition, which typically confers treatment resistance. Oxidative stress induced by Au@MIL treatments, including lipid oxidation in mitochondrial and endoplasmic reticulum, mitochondrial membrane potential changes, and ROS generation, has been evaluated. Lipid peroxidation, indicative of the oxidative breakdown of lipids, serves as a hallmark of ferroptosis. Rather than relying on iron, disrupting the redox balance of cancer cells with Au@MIL induces cell death, accompanied by notable lipid peroxidation. The alteration in mitochondrial membrane potential, suggestive of mitochondrial depolarization, indicates the initiation of apoptosis in the cells. Interestingly, no ROS is detected during the course of apoptosis. Notably, the introduction of a SiO₂ insulator between Au NPs and MIL directly blocks electron transfer from cancer cells, preventing the death of cancer cells. The manipulation of electron transfer pathways, particularly within cancer cells boasting hyperactive redox systems, presents a promising avenue for targeted therapies. Disrupting this delicate redox equilibrium holds potential for impeding the adaptive and growth mechanisms of cancer cells, offering a pathway for refined and effective therapeutic strategies against cancer.

The present disclosure provides a pharmaceutical composition comprising an effective amount of the gold nanoparticle composition as disclosed herein and optionally a pharmaceutically acceptable carrier. The pharmaceutical composition can optionally comprises one or more additives having a complementary therapeutic or diagnostic effect, wherein the additive is one selected from an antioxidant, an adjuvant, or a combination thereof.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, rectal administration, and direct injection into the affected area, such as direct injection into a tumor. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The present disclosure provides a method for treating cancer comprising administering the gold nanoparticle composition as disclosed herein to a subject in need. Alternatively, the present disclosure provides use of the gold nanoparticle composition as disclosed herein in the manufacture of a medicament for treating cancer in a subject in need.

In some embodiments, leveraging Shewanella oneidensis MR-1 membrane proteins, membrane-integrated liposomes abundant in c-type cytochromes are provided. These structures, when coupled with Au NPs, enable autonomous electron transfer from cancer cells, disrupting redox processes and inducing cell death. Our findings demonstrate efficacy across various cancer types, with larger Au NP size showing enhanced efficacy. The impact of using Au@MIL in cancer treatment is substantial, as it functions effectively under hypoxic condition. Oxidative stress induced by Au@MIL treatments, including lipid oxidation in mitochondrial and endoplasmic reticulum and mitochondrial membrane potential changes, initiates apoptosis, circumventing reliance on iron-mediated pathways. Surface plasmon band and X-ray absorption near-edge structure (XANES) investigations confirm electron transfer from cancer cells to Au NPs. Introduction of a SiO₂ insulator coating on Au NPs directly block electron transfer from cancer cells, suppressing cancer cells damaged. This innovative approach highlights the potential of modulated electron transfer pathways in targeted cancer therapy, offering a pathway for refined and effective treatments.

In some embodiments, the cancer is a solid cancer. Examples of the cancer include, but are not limited to, squamous cell cancer, lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, lymphomas, leukemias, myelomas and myeloproliferative neoplasms. In some embodiments, the cancer is liver cancer, lung cancer, pancreatic cancer, breast cancer, cervical cancer, or colorectal cancer.

The present disclosure provides a method for inducing lipid oxidation, membrane potential change and/or reactive oxygen species generation comprising administering the gold nanoparticle composition of as disclosed herein to a subject in need. Alternatively, the present disclosure provides use of the gold nanoparticle composition as disclosed herein in the manufacture of a medicament for inducing lipid oxidation, membrane potential change and/or reactive oxygen species generation in a subject in need.

In some embodiments, the lipid oxidation or membrane potential change occurs in mitochondrial or endoplasmic reticulum.

The following experiments and data are provided for illustrating the present disclosure.

### EXAMPLES

### Synthesis of Au NPs

Spherical Au NPs ranging from 15 to 100 nm were synthesized using different methods. Specifically, 15 nm Au NPs were synthesized using a modified Turkevich method, which involves the reduction of hydrogen tetrachloroaurate (III) trihydrate (HAuCl₄) with sodium citrate bihydrate at 120° C. Prior to conducting the syntheses, all glassware was thoroughly cleaned using aqua regia. Aqua regia was prepared by mixing one-part nitric acid with three parts hydrochloric acid. Initially, 1.8 mL of 5 mM HAuCl₄ was dissolved in 28.5 mL of ultrapure water and heated to 120° C. in an oil bath. Meanwhile, 1.22 mL of 38.8 mM sodium citrate bihydrate was freshly prepared in cold ultrapure water. Once the HAuCl₄ solution reached 120° C., the sodium citrate bihydrate solution was added dropwise with continuous stirring. Following the addition of sodium citrate bihydrate, the solution was stirred for an additional 10 minutes and then allowed to cool to room temperature while avoiding light exposure. The color of the mixture transitioned from pale yellow to wine red, indicating successful NP synthesis. The resulting 15 nm Au NPs, serving as the Au NP seeds, were harvested and stored at 4° C. in the dark until further use.

To synthesize larger Au NPs, ranging from 70 to 100 nm in diameter, a seeded growth method was employed. This method utilized 15 nm Au seeds as nucleation sites, and the final NP size was controlled by varying the amount of these seeds. The 15 nm Au seeds were prepared as previously described. For the synthesis of larger Au NPs, 0.5 mL of 50 mM HAuCl₄ was added to 100.5 mL of deionized water at room temperature. While rapidly stirring, the appropriate volume of 15 nm Au seeds (750μL for 70 nm, 300 μL for 100 nm) was added to the solution to achieve the desired sizes. Notably, greater volumes of 15 nm seeds produced smaller NPs. To initiate the NP growth reaction, 220 μL of 34 mM sodium citrate bihydrate was added to the solution, followed immediately by the addition of 1 mL of 0.03 M hydroquinone. The solution was allowed to stir at room temperature for three hours. Distinct color transitions were observed during the synthesis: the solution changed from black to wine-red for 70 nm Au NPs, and from black to red-brown for 100 nm Au NPs. Finally, the synthesized Au NPs (70 nm and 100 nm) were obtained by centrifugation for 10 minutes and washed twice with deionized water to remove residual reactants. The resulting Au NPs were re-dispersed in deionized water and stored at 4° C. in the dark until further use.

### Surface Pegylation of Au NPs

Au NPs were modified via PEGylation. Specifically, 5 mL of Au NP solution (Au concentration 400 ppm) was mixed with 1 mL of mSH-PEG (2 mg/mL) in double-distilled water (DDW) in glass vials and stirred at room temperature for 2 hours in the dark to modify the Au NP surface with mSH-PEG. After the reaction, the Au@PEG NPs were collected by centrifugation for 10 minutes at 15° C. and washed with DDW. This washing process was repeated at least twice. The resulting Au@PEG NPs were resuspended in DDW and stored at 4° C. in the dark until further use.

### Preparation of Silica-insulated Au NPs

The synthesis of silica-insulated Au NPs was carried out using a modified standard sol-gel method. Initially, 2 mL of Au NPs at a concentration of 1,500 ppm were mixed with 1 mL of NH₂-PEG-SH at 5 mg/mL. This mixture was then sonicated for 5 minutes in an ice bath. Following this, 6 mL of double-distilled water (DDW), 1 mL of ethanol (99.5%), 10 μL of NaOH (1 M), and 50 μL of tetraethyl orthosilicate (TEOS) were added sequentially, with sonication performed for 3, 3, 1, and 1 minutes, respectively, after each addition. The solution was then stirred vigorously for 18 hours in an ice bath to form SiO₂ coatings on the Au NPs. The product was collected by centrifugation for 3 minutes and washed at least three times with ethanol. Finally, the insulated Au NPs were dispersed in DDW for subsequent MIL encapsulation, resulting in the formation of Au@SiO₂@MIL NPs.

### Preparation of Defined Medium (DM)

The DM buffer solution was prepared by dissolving 1.25 g of NaHCO₃, 0.04 g of CaCl₂, 0.5 g of NH₄Cl, 0.1 g of MgCl₂, 5 g of NaCl, 3.6 g of HEPES, and 0.25 g of yeast extract in 0.5 L of deionized water. The mixture was autoclaved at 122° C. for 25 minutes to ensure sterility.

### Preparation of Liposomes

The individual stock solutions of 1,2-dioleoyl-sn-glycero-3-phosphocholine (DOPC) and 1,2-dioleoyl-sn-glycero-3-phosphoethanolamine (DOPE) in chloroform were prepared in sealed glass vials at a concentration of 10 mg/mL and stored at 4° C. A lipid mixture was then generated by combining DOPC and DOPE in a volume ratio of 0.504 mL: 0.118 mL. Subsequently, this mixture underwent nitrogen purging to achieve a multi-layered lipid film at the vial's bottom. The dried lipid film was rehydrated in 0.8 mL phosphate-buffered saline (PBS) to form liposomes with a composition of 70% DOPC and 30% DOPE. These liposomes were homogenized by extrusion through a polycarbonate filter with 100-nm pores. The resulting dispersed liposomes were stored at 4° C. for subsequent experiments.

### Bacterial Culture and MILs Preparation

Shewanella oneidensis MR-1 (obtained from ATCC) underwent cultivation on Luria broth (LB) agar at 30° C., yielding bacterial colonies. A single colony was subsequently inoculated into 80 mL fresh LB medium and incubated under slow shaking at 30° C. for 24 hours. Following this, the bacterial medium was substituted with defined medium (DM), adjusting the S. oneidensis MR-1 concentration to 4 optical density (O.D.). A 100 μL pre-prepared liposome solution was added to the 1 mL culture for further aerobic cultivation at 30° C. for 24 hours, during which MILs were predominantly produced through the LIME process (Chen, Y. C. et al. Nat. Nanotechnol. 18, 1492-1501 (2023); Li, W. P., Long, X., Kataoka-Hamai, C. & Okamoto, A. Membrane integrated liposome synthesized by a liposome fusion-induced membrane exchange. Preprint at https://doi.org/10.26434/chemrxiv- 2022-9tt9m (2022)). The resulting solution underwent centrifugation for 5 minutes at 4° C. to separate bacteria and MILs. Supernatants containing MILs and residual S. oneidensis MR-1 were filtered using a 0.45 μm syringe filter to remove remaining bacteria. The MIL suspensions were then ultracentrifuged at 4° C. for 4 hours. The resulting MIL pellet was resuspended in DM and stored at −20° C. for subsequent experiments.

Wild-type Escherichia coli(E. coli) was obtained from ATCC. For E. coli culture, LB medium containing 50 mg/L kanamycin was used, and the culture was maintained at 30° C. The process for preparing MILs was identical to that used for S. oneidensis MR-1, resulting in the production of E. coli -derived MIL.

### Protein Quantification

The protein concentration was determined in accordance with the manufacturer's instructions using a Bio-Rad protein assay kit (Bio-Rad Laboratories, Hercules, CA, USA). Bovine serum albumin (BSA) solutions were employed to establish the standard calibration curve. Initially, samples were combined with radioimmunoprecipitation assay buffer (RIPA) and incubated for 10 minutes. Following this, the supernatants containing proteins were obtained through centrifugation for 5 min. Each sample (4 μL) was then mixed with 50 μL of Bio-Rad protein assay reagent (reagent A/reagent S 50:1), followed by the addition of 400 μL of reagent B (as labeled by the manufacturer) for 5 minutes. Later, 200 μL of supernatants or standard solutions were transferred to a 96-well plate, and the optical density (O.D.) at 690 nm for each well was measured using a microplate reader (800 TS, BioTek, USA).

### Preparation of Membrane-Coated Au NPs

The combination of Au NPs (100 ppm) and MILs (1 mg/mL) underwent sonication (DELTA-DC 80, 40 kHz, 80 W, and 10 min) to disintegrate MILs. Subsequently, the mixture was rapidly frozen in an ice bath for 5 minutes to facilitate the integration of MIL components onto the surface of Au NPs, resulting in the formation of Au@MIL NPs. This sonication-cooling process was iterated three times to optimize membrane coverage on Au NPs. The resulting Au@MIL NPs were then collected through centrifugation for 5 minutes and subjected to three washes with PBS. The same procedure was followed to obtain the Au@Lipo NPs and Au@E. coli NPs.

### Protein Electrophoresis

The protein electrophoresis system (Hoefer SE260) was set up with TGS running buffer in the tank and a commercial PAGE gel installed. Samples were prepared by mixing 20 μL of bacterial extract, MILs, Au@PEG, or Au@MIL with 4 μL of 6X Laemmli SDS sample buffer and incubating at 95° C. for 5 minutes. A 15 μL sample was loaded into each well of the PAGE gel, and electrophoresis was run at 120 V for approximately 40 minutes. Gels were stained with Brilliant Blue R for whole protein visualization.

For heme-staining, gels were prepared similarly but using a 2-mercaptoethanol-free 4X LDX sample buffer to replace the 6X Laemmli SDS sample buffer. Staining was done in a 50 mL solution containing 6.3 mM TMBZ and 0.25 M sodium acetate (pH 5.0) in the dark for 2 hours, followed by the addition of 47.5 μL of 30% hydrogen peroxide and incubation for 30 minutes. The gel background was destained using a solution of 30% isopropanol and 70% sodium acetate.

### Cultivation and Treatment of Cancer and Normal Cell Lines

A diverse array of cancer cell lines, including Huh-7, Hep G2, SK HEP-1, HA22T, A549, Hep 3B, AsPC-1, PANC-1, MDA-MB-231, HeLa, and T24, along with the normal cell lines NeHepLxHT and M10, were propagated in media formulations comprising high-glucose DMEM, MEM, and RPMI. These media were supplemented with 10% fetal bovine serum (FBS), 1% penicillin/streptomycin (P/S), and 1% non-essential amino acids (NEAA), all sourced from Caisson (Smithfield, VA, USA). Additionally, human neonatal hepatocyte cells (NeHepLxHT) (Reid, Y., Gaddipati, J. P., Yadav, D. & Kantor, J. In Vitro Cell Dev. Biol. Anim. 45, 535-542 (2009); Li, T. N. et al. Intrahepatic hepatitis B virus large surface antigen induces hepatocyte hyperploidy via failure of cytokinesis. J. Pathol. 245, 502-513 (2018)), genetically modified to express the hTERT gene, were cultured in high-glucose DMEM/F-12 enriched with 10% FBS, 1% P/S, 100 nM dexamethasone, 0.1% ITS premix, 20 ng/mL human epidermal growth factor (EGF), and 5 mg/mL ciprofloxacin. All cell cultures were incubated at 37° C. within a humidified atmosphere containing 5% CO₂.

To simulate hypoxic conditions, a specialized hypoxia chamber was utilized, maintaining an atmospheric composition of 1% O₂, 5% CO₂, and 94% N₂ within the incubator. After 48 hours of hypoxic exposure, cellular specimens were harvested for further examination.

### Visualization of Cellular Cytotoxic and Oxidative Stress Responses

To visualize cytotoxicity in cancer treatments, cancerous and normal cells (30,000 cells per well) were seeded into 24-well plates and cultured for 24 hours. The cells were then treated with Au@PEG, Au@Lipo, Au@E. coli, or Au@MIL NPs (Au concentration of 300 ppm) and incubated for an additional 48 hours for 100 nm NPs or 72 hours for 70 nm NPs. After NPs internalization, the medium was carefully aspirated, and the cells were washed three times with PBS to remove non-internalizing NPs. The cells were subsequently stained with 2 μg/mL Hoechst 33342 for nuclei, 1 μM calcein AM for live cells, and 1 μM EthD-1 for dead cells. Fluorescence images were captured using a 20× objective lens on an Olympus IX71 fluorescence microscope.

To examine intracellular oxidative stress, Hep G2 cells were seeded in 24-well plates and cultured overnight. The positive control group was treated with 500 μM tert-butyl hydroperoxide (tBHP) for 3 hours. The other groups were treated with Au@MIL or Au@E. coli NPs (Au concentration of 300 ppm) and incubated for an additional 48 hours. Following the incubation period, all cells were washed three times with PBS and stained with 10 μM DCFH-DA fluorescent dye to determine intracellular ROS levels.

### Detection of Intracellular Atp Production

Hep G2 cells (30,000 per well) were seeded into 24-well plates and cultured for 24 hours. The cells were then treated with Au@MIL medium solution (100 nm Au of 300 ppm), with untreated cells serving as the control group. After 48 hours of incubation, the cells were incubated with the fluorescent probe ATP Red-1 (10 μM, Sigma-Aldrich) at 37° C. for 15 minutes to detect mitochondrial ATP production. Fluorescence images were captured using a 20× objective lens on a fluorescence microscope (IX71, Olympus, Tokyo, Japan) and fluorescence intensity was measured using ImageJ software (National Institutes of Health, MD).

### Hemolysis Analysis

A 2% suspension of red blood cells was introduced into various solutions, including deionized water (positive control group), PBS (negative control group), PBS with Au@PEG NPs, or PBS with Au@MIL NPs, with an Au concentration of 50 ppm. The sample solutions were incubated at room temperature in the dark for 1 hour. Subsequently, hemoglobin quantification in the supernatants of different treatments was determined by measuring the absorbance of hemoglobin at 450 nm using a microplate reader. After completing the assay, the percentage of hemolysis was calculated using the following equation:

$$Percentage of Hemolysis = ( {Absorbance}_{sample} - {Absorbance}_{control} ) / ( {Absorbance}_{maximal lysis} - {Absorbance}_{control} ) ] \times 100 \qquad ( 1 )$$

### Clonogenic Assay

One thousand cells were seeded in a 6-well culture plate overnight, then treated with 300 ppm Au-based NPs, including Au@MIL (70 nm and 100 nm), Au@PEG (70 nm), Au@Lipo, and Au@E. Coli, for 72 hours. After refreshing the cells with medium without NPs, they were further incubated for another 14 days. The formation of colonies was fixed with methanol for 30 minutes, stained with 0.5% crystal violet for another 30 minutes, and then measured using ImageJ software (version 1.53r).

### Rna Sequencing and Bioinformatics Analysis

Total ribonucleic acid (RNA) was extracted from cancer cells treated with PBS and 300 ppm Au@MIL (100 nm) using Trizol Reagent (#15596026, THERMO FISHER SCIENTIFIC®). For RNA library preparation, the refined RNA underwent processing with the TruSeq™ Stranded mRNA Library Prep Kit from ILLUMINA®, San Diego, CA, USA, following the manufacturer's guidelines.

In summary, mRNA (1 μg) was isolated from total RNA using oligo(dT)-coupled magnetic beads and subsequently fragmented at an elevated temperature. The first-strand cDNA was synthesized using reverse transcriptase and random primers. After the generation of double-strand cDNA and adenylation at the 3′ ends of DNA fragments, adaptors were ligated and purified using the AMPure XP system from Beckman Coulter, Beverly, USA. The library quality was assessed using the Agilent™ Bioanalyzer 2100 and Real-Time PCR systems. Subsequently, the qualified libraries were sequenced on an ILLUMINA® Novaseq X plus platform, generating 150 bp paired-end read.

The fastp program (version 0.20.0) was employed to eliminate low-quality bases and sequences originating from adapters in the raw data. The processed reads were aligned to the reference genomes using HISAT2 (version 2.1.0). The FeatureCounts software (v 2.0.1) within the Subread package was utilized for gene abundance quantification. Differentially expressed genes (DEGs) were identified using either DESeq 2 (version 1.28.0) or EdgeR (version 3.36.0), depending on the presence or absence of biological replicates. The R package clusterProfiler (version 4.0.0) was employed to conduct functional enrichment analysis of gene ontology (GO) within gene clusters.

### JC-1 Staining

1×10⁵ cancer cells were seeded in 6-well overnight and treated with PBS, and 300 ppm Au@MIL (100 nm). After 24, 48, and 72 hours post-treatment, the cancer cells were stained with a JC-1 mitochondrial membrane potential assay kit (HY-K0601, MEDCHEMEXPRESS®) according to the manufacturer's guidelines. The images were observed using an inverted fluorescence microscope (CKX53, OLYMPUS®), excited at 510 nm and emitted at 527 nm (green) and 590 nm (red). Three different fields were taken for each group and analyzed using ImageJ software (version 1.53r).

### Measurement of Lipid ROS in Hep G2 Cells

Lipid peroxidation in Hep G2 cells was assessed using the C 11-BODIPY 581/591 fluorescence probe (THERMOFISHER®, Massachusetts, USA) according to the manufacturer's instructions. Hep G2 cells were seeded at a density of 4,000 cells per well in 96-well glass-bottom microplates (GREINER®, Austria). The cells were treated with Au@MIL (100 nm Au of 300 ppm) for 24 hours to induce oxidation. Additionally, cells treated with Ferroptocide (50 μM) for 1 hour served as a positive control, while untreated cells were used as the control group. All groups were gently washed twice with PBS to remove non-internalizing NPs and then treated with 10 μM C11-BODIPY 581/591 solution and 4 μg/mL Hoechst 33258 (MERCK®, USA) at 37° C. for 1 hour. Finally, the cells were washed with PBS and imaged using an ImageXpressMicro (IXM) high-content imaging system operated by MetaXpress software version 6.7.0.211.

### Trafficking of Lipid Peroxidation by Au@MIL NPs

To study the lipid peroxidation induced by Au@MIL NPs, fluorescent protein RFP-tagged proteins (mitochondrial Cox8-mRFP and the endoplasmic reticulum ERp57-RFP) were used to label mitochondria and the endoplasmic reticulum, respectively. Hep G2 hepatoma cells were seeded at a density of 5×10⁵ cells per dish in 3.5 cm glass-bottom dishes and incubated for 24 hours. The cells were then transfected with Cox 8-mRFP and ERp57-RFP plasmids using Lipofectamine 3000 (THERMO FISHER SCIENTIFIC®) and incubated for an additional 24 hours. Subsequently, cells were treated with Au@MIL NPs for 24 hours. After another 24 hours of incubation, the cells were rinsed with fresh phenol red-free medium. Images were obtained using a confocal microscope (FV3000, OLYMPUS®) equipped with a 100× oil lens.

### Mice

Animal care adhered to the Laboratory Animal Welfare Act and the Guide for the Care and Use of Laboratory Animals. The experimental mice, including NOD-SCID and C57BL/6 strains, were housed in cages with three to five mice per cage. They were maintained at 22-23° C. with 55±10% humidity, following a 13 hours/11 hours light/dark cycle.

### Establishment of the Hep G2-red-fluc Orthotopic Hcc Xenograft Mice

NOD-SCID mice (male, 6-8 weeks old) were anesthetized with Zoletil 100 (Virbac), administered intraperitoneally and placed supine. Subsequently, 2×10⁶ Hep G2-Red-Fluc cells in a solution comprising 10 μL of PBS and 10 μL of Basement Membrane Matrix (BD) were surgically implanted into either the right or left lobe of the liver using BD® Insulin Syringes 30 G 3/10cc (BD®). The incision was closed using CT 204 Chromic Catgut (20 mm, 75 cm, UNIK SURGICAL SUTURES MFG. CO.) and NC193 Monofilament Nylon (19 mm, 45 cm, UNIK SURGICAL SUTURES MFG. CO.). Subsequently, the mice were allowed to recover fully.

### Evaluation of Anti-Tumor Efficacy and Biodistribution of Au@MIL in Vivo

Hep G 2-Red-FLuc orthotopic HCC xenograft mice were treated with 100 μL of sterilized PBS, 100 μL of 3000 ppm Au@PEG (100 nm), and 100 μL of 3000 ppm Au@MIL (100 nm) through intravenous administration for a single dose. The body weight of each group was recorded twice a week, and tumor growth in Hep G2-Red-FLuc hepatocellular carcinoma cells was monitored using the IVIS imaging system (CALIPER LIFE SCIENCES®). For sample collection, all mice were sacrificed on Day 14 post-treatment. For survival analysis, the Institutional Animal Care and Use Committee stipulated that the maximal tumor burden should not exceed 10% of the body weight, and ascites formation should be absent. All experimental mice were sacrificed before reaching these specified limits.

For biodistribution analysis, samples of the heart, lung, spleen, kidney, liver, HCC tumor, and feces were collected from Au@MIL-treated HCC mice at 1, 4, 8, 24, and 72 hours post-treatment. The samples were disrupted into powder and acid-digested in aqua regia for one week. The concentration of Au in the sample solution was determined by iCAP™ 7400 ICP-OES (THERMO FISHER SCIENTIFIC®).

### Ivis Imaging System and Quantification

The mice were anesthetized using a combination of oxygen and isoflurane and subsequently intraperitoneally administered 100 μL of D-luciferin (catalog #122796, CALIPER LIFE SCIENCES®). Ten minutes later, the mice were imaged using the Xenogen IVISR Spectrum Noninvasive Quantitative Molecular Imaging System (CALIPER LIFE SCIENCES®) and analyzed with Living Image 4.7.3 (PERKINELMER®, USA). The liver was collected and subjected to ex vivo IVIS detection after the mice's sacrifice.

### Biosafety of Au@mil in Vivo

C57BL/6 mice (male, 6-8 weeks old) were treated with 100 μL of sterilized PBS and 100 μL of 3000 ppm Au@MIL (100 nm) through a single intravenous administration. The body weight of each group was recorded daily, and all mice were sacrificed on Day 7 post-treatment.

### Hematoxylin and Eosin (H&E) Staining

Tumor and standard organ samples, including the heart, lung, spleen, liver, and kidney, were embedded in paraffin and sliced into 5 μM thickness. The sections underwent deparaffinization, rehydration, and PBS washing, then staining with hematoxylin solution for 3 minutes. After washing in tap water, eosin solution was applied for 1 minute. Subsequently, the sections were immersed in ethanol and xylene before being mounted for evaluation. The sections were examined under a microscope (CX31, OLYMPUS®), and three fields were captured for each group.

### Immunohistochemistry (IHC) Staining

Tumor samples were embedded in paraffin and sliced into 5 μm thickness. The sections underwent deparaffinization, rehydration, and incubation with cleaved caspase-3 (Asp175) antibody (1:400 dilution, #9661, CELL SIGNALING TECHNOLOGY®) and 4-Hydroxynonenal (1:200 dilution, #bs- 6313R, BIOSS®). Staining was carried out using an ABC peroxidase standard staining kit (THERMO FISHER SCIENTIFIC®) containing biotinylated affinity-purified goat anti-rabbit IgG (1:1000 dilution, #32054, THERMO FISHER SCIENTIFIC®) and a DAB peroxidase (HRP) substrate kit (VECTOR LABORATORIES®) as per the manufacturer's protocol. Finally, the sections were examined under a microscope (CX31, OLYMPUS®), with three different fields captured for each group.

### Biochemical Analysis

Blood was extracted from the mice's hearts, and heparin sodium was promptly added. The collected blood samples underwent centrifugation for 10 minutes to obtain the serum. Subsequently, the serum samples were utilized for blood biochemistry analysis, including alanine aminotransferase (ALT), alkaline phosphatase (ALP), aspartate aminotransferase (AST), total bilirubin (T-Bil), blood urea nitrogen (BUN), creatine (CREA), and uric acid (UA) expression. The analysis was performed using a FUJI DRI-CHEM 4000i (FUJIFILM®). For urine analysis, the collected samples were analyzed using the Thinka Urine Test Strip 10UB (ARKRAY®) with the RT-4010 analyzer (ARKRAY®).

### XANES Measurement

The XANES spectra were measured at TLS 01C1 beamline. XANES measurements at the Au L3-edge were conducted in fluorescence mode using a Lytle detector. The spectra of Au foil were collected in transmission mode for comparison and monochromatic energy calibration. The acquired XANES data were processed according to the standard procedures using the Athena program (Ravel, B. & Newville, M. ATHENA, ARTEMIS, HEPHAESTUS: data analysis for X-ray absorption spectroscopy using. J. Synchrotron Radiat. 12, 537-541 (2005)).

### Statistical Analysis

Statistical analysis was performed using MICROSOFT® Excel 2016, or Origin software version 8.1 for WINDOWS®. Student's t-test or one-way ANOVA was employed to determine statistical significance, with a p-value less than 0.05 considered statistically significant.

### Example 1 Synthesis and Characterization of Au@MIL NPs

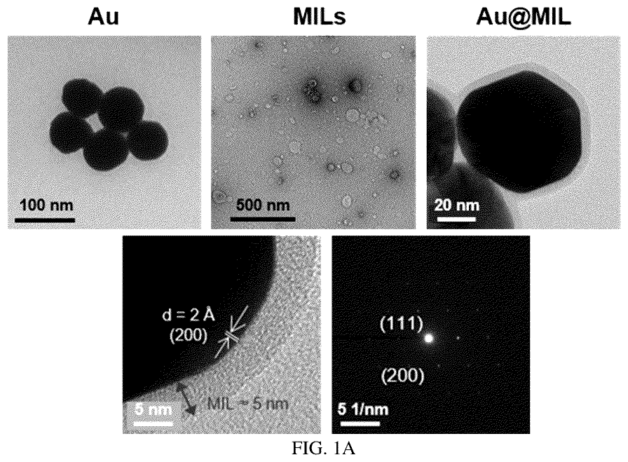

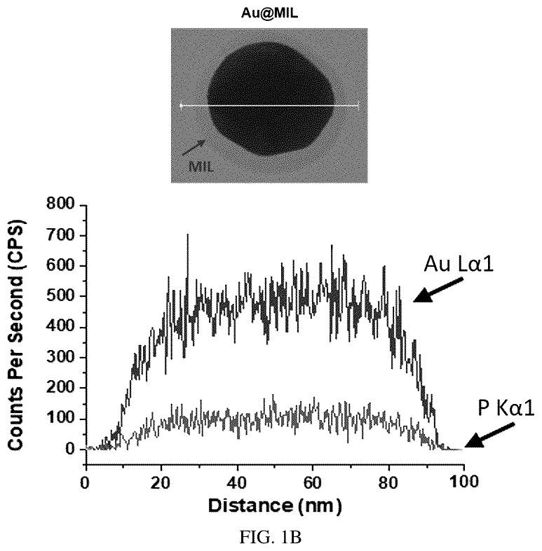

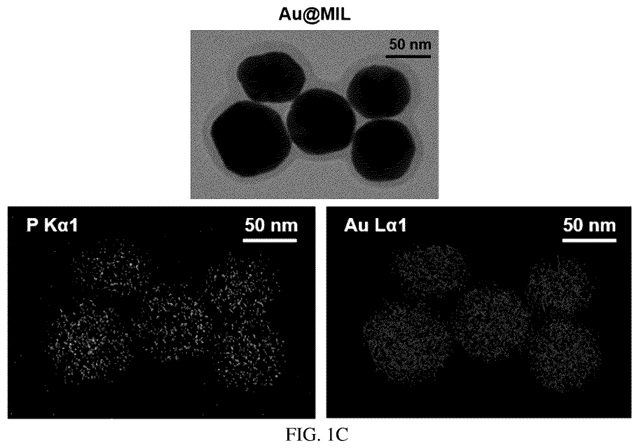

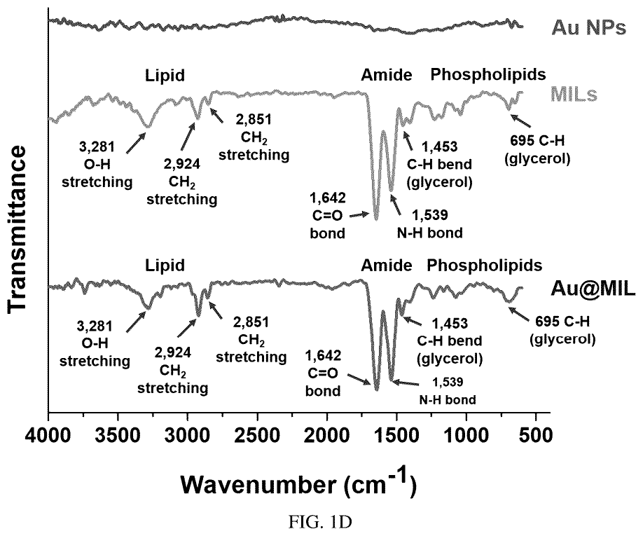

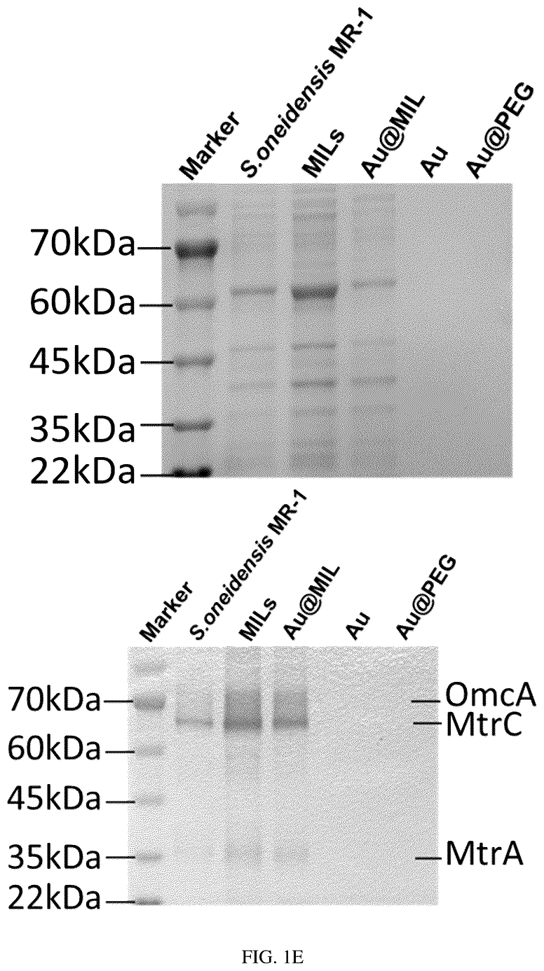

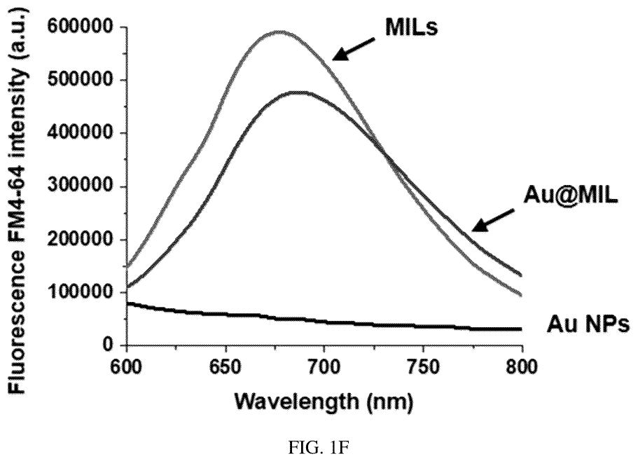

Au NPs with an average size of 70 nm were synthesized using a modified Turkevich method (Dong, Y. C. et al. Sci. Rep. 9, 14912 (2019)) (FIG. 1A). Following our developed technique termed LIME, vesicular structures known as membrane-integrated-liposomes (MILs) were efficiently produced from Shewanella oneidensis MR-1 (FIG. 1A). During the sonication-cooling process, the MILs deconstructed and subsequently self-assembled onto the surface of the Au NPs, forming MIL-coated Au NPs (Au@MIL NPs; FIG. 1A). High-resolution TEM (HRTEM) images clearly show the MIL membrane adhering to the surface of the Au NPs, with a thickness of approximately 5 nm, closely correlating with that of a single-layer cell membrane. The lattice spacing of the (200) plane in the face-centered cubic (FCC) structure of Au was observed to be 2 Å (ICDD PDF card no. 00-004-0784). Additionally, the electron diffraction pattern indicates a crystalline structure of Au@MIL, corresponding to the FCC structure of Au with (111) and (200) planes, suggesting no structural change after membrane coating.

To optimize the MIL coating on the Au NPs, different ratios of MIL to Au were used during the sonication-cooling process. The resulting Au@MIL NPs were then analyzed for hydrodynamic diameter and zeta potential. The hydrodynamic diameter increases gradually with higher MIL/Au ratios, reaching a maximum value at a ratio of 10. Conversely, the zeta potential values decrease as the MIL/Au ratio increased, reaching −13.1 mV, which is close to the value of MIL alone. This indicates complete coverage of the MIL on the Au NPs at a ratio of 10. Based on both hydrodynamic diameter and zeta potential analyses, a MIL/Au ratio of 10 is determined to be optimal for fabricating Au@MIL NPs.

FIGS. 1B and 1C present the energy dispersive spectroscopy (EDS) signal of the phosphorus element around the Au NPs, attributed to the phosphate lipid component in MIL, confirming the presence of the Au core and MIL shell. Furthermore, FT-IR measurements provide additional evidence of MIL on the Au NPs (FIG. 1D). For MILs, characteristic peaks were detected in the regions of 690-1200 cm⁻¹, 1500-1700 cm⁻¹, and 2850-3300 cm⁻¹, corresponding to the phospholipid components, amino acids from membrane proteins, and the hydrocarbon skeleton and hydroxyl groups in lipids, respectively. These findings indicate the successful integration of MIL onto the Au NPs.

### Example 2 Characterization of Membrane on the Au@MIL NPs

Characterizing the membrane proteins on the Au@MIL NPs is crucial to ensure the functionality of cytochromes in facilitating electron transfer from cancer cells to Au NPs. Sodium dodecyl sulfate-polyacrylamide gel electrophoresis (SDS-PAGE) was used to separate proteins by molecular weight, followed by staining to identify the proteins on the gel, providing evidence of proteins on the Au@MIL NPs. As expected, both MILs and Au@MIL NPs stained with Coomassie Brilliant Blue (CBB) reveal the presence of membrane proteins extracted from S. oneidensis MR-1 (FIG. 1E). Heme staining further identified representative heme-based cytochromes, including MtrA, MtrC, and OmcA, from S. oneidensis MR-1, MILs, and Au@MIL NPs (FIG. 1E). This analysis confirms the successful coating of MIL on Au NPs without protein loss. No proteins were detected in the Au NPs alone or in Au@PEG NPs. Polyethylene glycol thiol (mPEG-SH, MW=3,400) was used to modify the Au NPs as a negative control for comparison.

The concentration of total proteins on Au@MIL NPs was further quantified using a standard protein assay. The protein amounts on Au@MIL NPs increase gradually as a function of the MIL/Au ratio, with an optimal ratio of 10 achieving the maximum cytochrome content. The presence of cytochromes in MILs, constructed from heme proteins, allows the concentration of heme to serve as an index for determining the cytochrome content. Due to the specific structure of heme, which contains a porphyrin ring chelating an Fe center, heme concentration can be determined by measuring Fe concentration. Fe concentration in Au@MIL NPs was measured using ICP-OES (iCAP™7400). In a 3.5 mL solution containing 0.96 mg of Au@MIL NPs, the Fe concentration was found to be 0.065 ppm. Consequently, the mass of Fe, the moles of Fe, and the number of Fe atoms were calculated to be 2.27×10⁻⁷g, 4.07×10⁻⁹ moles, and 2.45×10¹⁵ atoms, respectively. Given that MtrC, MtrA, and OmcA are part of the decaheme cytochromes, the maximum amount of decaheme cytochrome was determined to be 2.55×10¹⁴ per 1 mg of NPs. The density of Au NPs was estimated to be approximately 19.32 g/cm³, assuming the volume of Au NPs is close to that of a sphere with a 70 nm diameter. The mass and number of Au NPs were calculated to be 3.47×10⁻¹⁵ g and 2.88×10¹¹ NPs per mL, respectively, corresponding to 887 decaheme cytochromes per Au NP. To provide additional evidence for the presence of the membrane on the NPs, a membrane-specific fluorescent dye, FM 4-64, which emits at 680 nm, was utilized (FIG. 1F). Fluorescence signals were detected in both MILs and Au@MIL NPs, indicating the presence of the membrane. No fluorescence emission was observed in the bare Au NPs.

### Example 3 In Vitro Cytotoxicity Evaluation using 70 nm Sized Au in Au@MIL NPs

To investigate the anti-cancer efficacy of Au@MIL, thirteen different cell lines were selected for testing. The study includes two normal cell lines and eleven cancer cell lines, utilizing the LIVE/DEAD cell imaging kit assay to determine the cytotoxic effects of the 70 nm Au@MIL NPs. Live and dead cells were stained with calcein AM and ethidium homodimer-1 (EthD-1), respectively, followed by treatment with 70 nm Au@MIL NPs for imaging evaluation. After 72 hours of incubation, no cell death signal was observed in the normal cell lines (NeHepLxHT, M10). In contrast, substantial damage was observed in the cancer cell lines post-treatment with Au@MIL NPs after the same incubation period. These results indicate a significant reduction in the viability of cancer cells incubated with 70 nm Au@MIL NPs, suggesting that Au@MIL can be broadly applied across different types of cancer.

Having demonstrated the cytotoxic effect of 70 nm Au@MIL NPs on various cancerous cells, we selected five cell lines for more detailed investigation: two normal cell lines (NeHepLxHT and M10) and three cancerous cell lines (Hep G2, HA22T, and MDA-MB-231). To clarify the role of MIL in targeting cancerous cells, we prepared Au NPs with different coatings: Au@PEG, Au@Lipo (liposome), and Au@E. coli (Escherichia coli membrane) for comparison with Au@MIL regarding therapeutic efficacy. The membrane of E. coli lacks the necessary proteins to facilitate electron transfer, making it a relevant control. We followed the standardized LIME method to extract the membrane from E. coli, forming Au@E. coli.Protein-free liposome-coated Au NPs (Au@Lipo) were also prepared for evaluation. Notably, no cytotoxic effect was observed in either cancerous or normal cells treated with Au@PEG, Au@Lipo, or Au@E. coli.

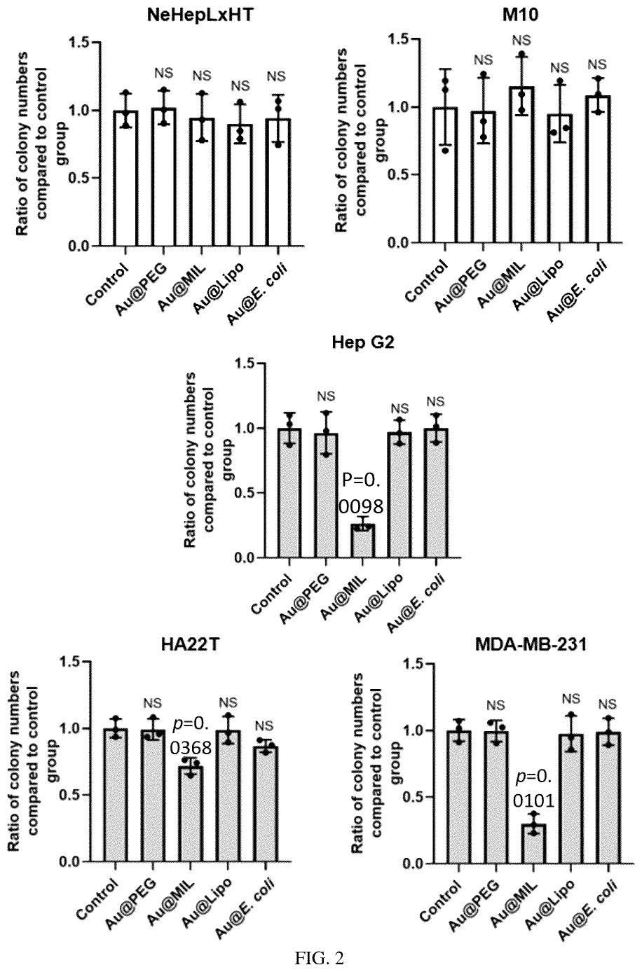

The colony assay quantified the cytotoxicity of 70 nm Au@MIL NPs across the five selected cell lines. Specifically, there is approximately 74% inhibition in Hep G2 cells, around 29% inhibition in HA22T cells, and about 70% inhibition in MDA-MB- 231 cells. In contrast, no cytotoxicity was observed in the normal cell lines NeHepLxHT and M10 (FIG. 2). Control groups containing Au@PEG, Au@Lipo, and Au@E. coli showed no toxicity to either cancerous or normal cell lines. These findings underscore the critical role of electron transfer facilitated by MILs, which is essential for inducing cytotoxic effects specifically in cancer cells.

### Example 4 Size-Dependent Efficacy of Au@mil Nps in Cancer Cell Eradication

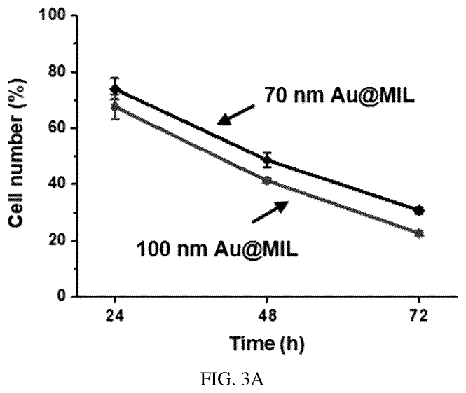

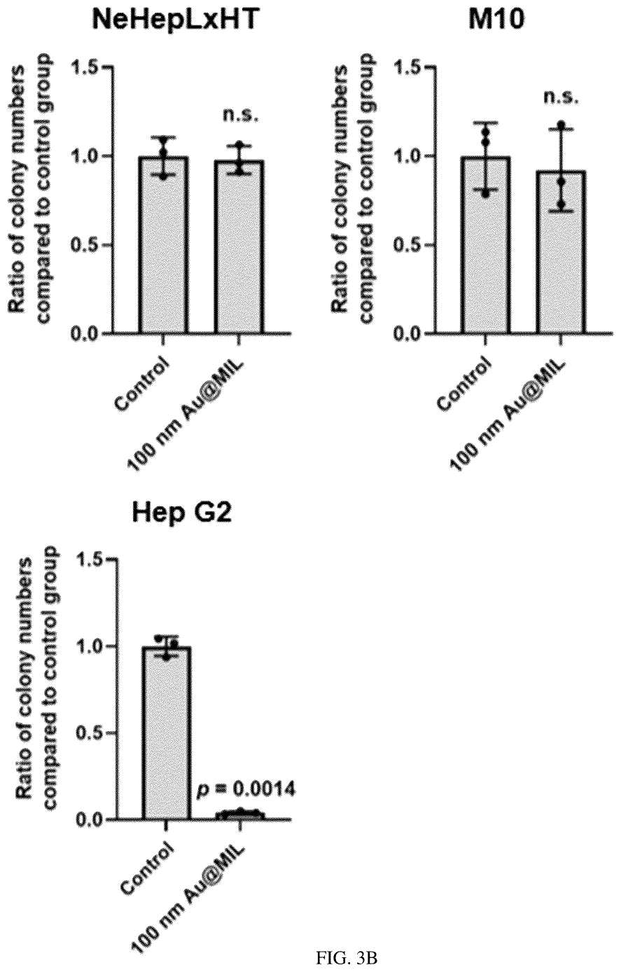

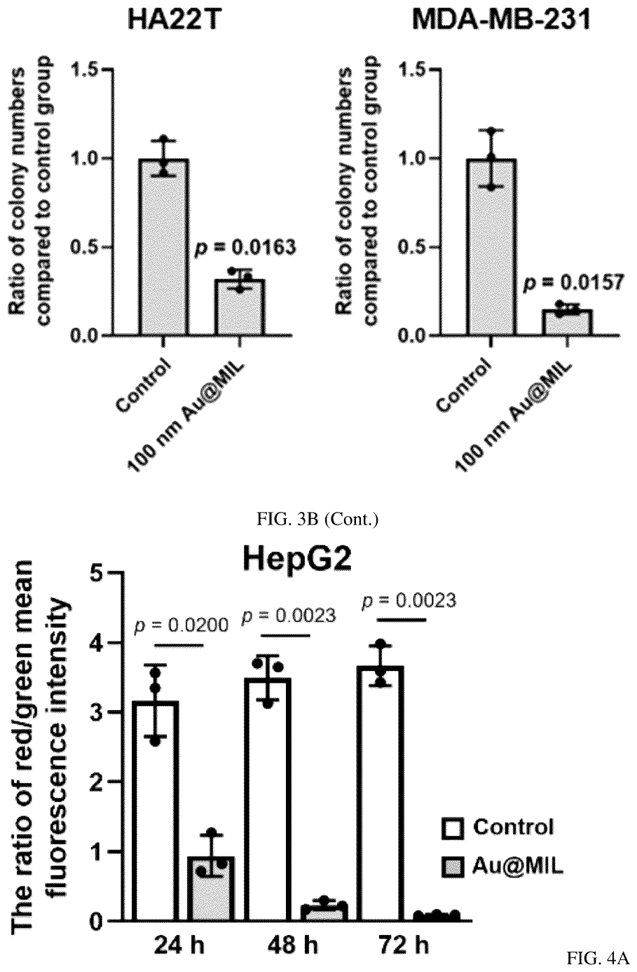

The discovery that 70 nm sized Au NPs can kill cancer cells has sparked interest from a nanomaterials perspective in understanding whether this effect is size-dependent. To explore this, larger Au NPs (100 nm) were used to study their efficacy in eradicating cancer cells compared to smaller 70 nm size in forming Au@MIL NPs. Initially, the effect of NP size was examined using Hep G2 cells to evaluate their electron attraction capacity. Live and dead cell observations illustrated in FIG. 3A show Hep G2 cell viability when treated with 70 nm and 100 nm sizes at 300 ppm for 24, 48, and 72 hours. A size-dependent behavior in cell viability was observed, with 100 nm Au@MIL NPs inducing greater Hep G2 cell death compared to 70 nm NPs at the same concentration. Thus, further studies involve treating two normal cell lines (NeHepLxHT, M10) and three cancer cell lines (Hep G2, HA22T, and MDA-MB- 231) with 100 nm Au@MIL NPs at 300 ppm for 48 hours. Once again, no cell death was observed in the normal cell lines, while significant cell damage was seen in the cancer cell lines post-treatment. The colony assay further demonstrated significant anti-cancer cytotoxicity of 100 nm Au@MIL NPs across cancer cell lines, with approximately 96% inhibition in Hep G2 cells, 68% inhibition in HA22T cells, and 85% inhibition in MDA-MB- 231 cells (FIG. 3B). These results highlight a significant size-dependent anti-cancer effect of Au@MIL NPs.

Prior to further in vitro examination, we conducted RNA sequencing analysis on Hep G2 and MDA-MB- 231 cells treated with 100 nm Au@MIL NPs (N), chosen based on greater anti-cancer efficacy, for 24 hours, comparing them to untreated control groups (C) to comprehensively explore regulatory mechanisms. We identified 8230 significantly differentially expressed genes in the Hep G2 groups (H_C vs. H_N) and 6100 in the MDA-MB-231 groups (M_C vs. M_N), with an overlap of 5032 genes. Gene Ontology (GO) enrichment analysis reveals that these intersected genes are involved in various biological processes, including DNA repair, mitochondrial transition, cell cycle regulation, oxidative stress response, and apoptotic processes. They are associated with cellular components such as the cytosol, mitochondrion, nucleus, cytoplasm, and membrane, and are linked to molecular functions such as RNA, ATP, DNA, protein, and metal ion binding. Given the importance of electron transfer from cancer cells to Au NPs, our follow-up studies focus on mitochondrial function and oxidative stress.

### Example 5 Au@MIL NPs-Induced Mitochondrial Dysfunction

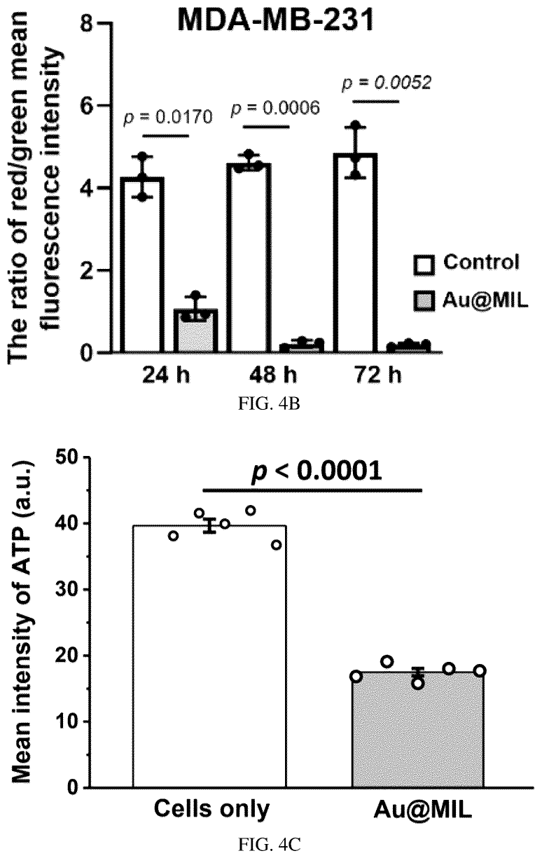

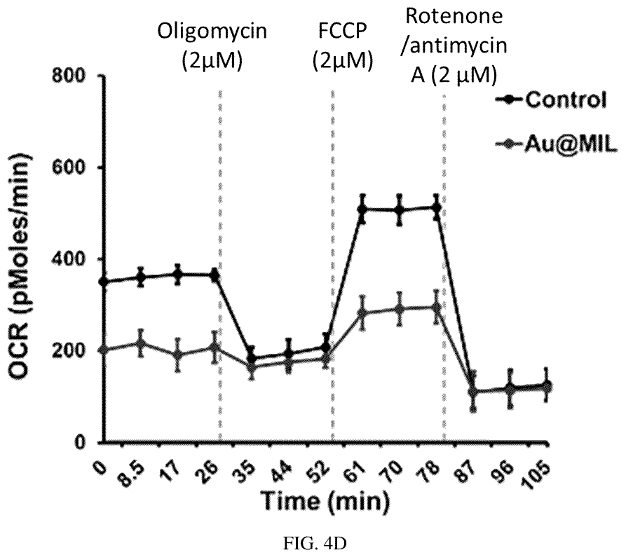

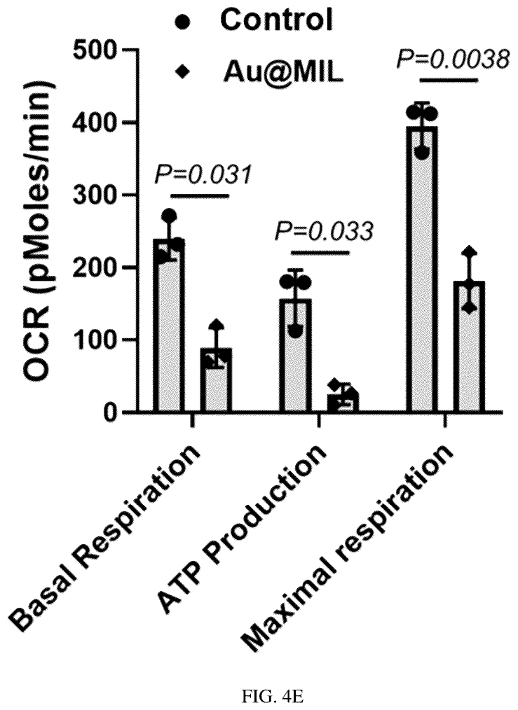

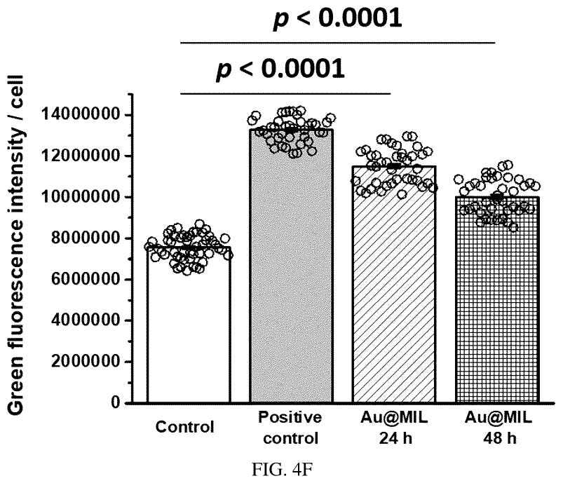

Mitochondria are crucial organelles that regulate cellular energy balance, oxidative stress responses, and cell survival. To investigate the mechanism of cell death induced by Au@MIL NPs, we used the dual-emissive fluorescent probe JC-1 to assess mitochondrial membrane potential. High membrane potential leads to red fluorescence due to mitochondrial J-aggregates formation, while low potential results in green fluorescence from cytosolic J-monomers. Mitochondrial dysfunction, indicative of decreased membrane potential, was assessed by a decreased ratio of red-to-green fluorescence intensity. In both Hep G2 and MDA-MB-231 cancer cells (FIGS. 4A and 4B), the Au@MIL-treated groups exhibited a lower red-to-green fluorescence ratio compared to the control, indicating increased mitochondrial depolarization. Furthermore, mitochondrial depolarization intensified with longer exposure times (24, 48, and 72 hours), underscoring a time-dependent effect of Au@MIL NPs. Additionally, ATP-Red 1, a mitochondrial ATP fluorescence probe, was used to investigate ATP depletion induced by Au@MIL NPs. Our results demonstrate a significant reduction in ATP synthesis in the Au@MIL-treated groups compared to controls (FIG. 4C). Moreover, the XF Cell Mito Stress Test indicates defects in mitochondrial function in Hep G2 cells treated with Au@MIL for 24 hours. These defects include impairments in basal respiration, ATP production, and maximal respiration (FIGS. 4D and 4E). Therefore, our findings highlight that Au@MIL NPs lead to diminished ATP production and loss of mitochondrial membrane potential.

### Example 6 Au@mil NPs-Induced Lipid Peroxidation

Lipid peroxidation, a critical marker of ferroptosis, involves the oxidative degradation of lipids due to disruption in cellular redox balance. To detect lipid peroxidation, the lipophilic fluorescent probe BODIPY™ 581/591 C 11 was utilized, which shifts its fluorescence emission from red (590 nm) to green (510 nm) upon oxidation. As a positive control for inducing rapid ferroptotic cell death, Ferroptocide was employed. Confocal microscopy images of Hep G2 cells reveals an increase in green fluorescence following treatment with Au@MIL NPs, indicating heightened lipid peroxidation compared to untreated cells (FIG. 4F). To further elucidate the localization of lipid peroxidation, mitochondrial Cox8-mRFP and endoplasmic reticulum ERp57-RFP markers were employed in conjunction with BODIPY™ 581/591 C11 staining. Our findings confirm that Au@MIL NPs induced lipid peroxidation in both mitochondria and the endoplasmic reticulum.

### Example 7 Absence of Intracellular Ros by Au@mil NPs

Intracellular ROS production induced by Au@MIL NPs was assessed using the ROS probe 2′,7′-dichlorodihydrofluorescein diacetate (DCFH-DA), which emits green fluorescence upon oxidation. Tert-butyl hydroperoxide (tBHP) served as a positive control for inducing oxidative stress. However, no ROS production was observed in the control, Au@MIL, and Au@E. coli groups. This indicates that Au@MIL NPs do not induce the production of intracellular ROS, specifically hydrogen peroxide (H₂O₂), hydroxyl radicals (•OH), peroxyl radicals (ROO•), or peroxynitrite (ONOO⁻). Overall, the cytotoxicity of Au@MIL NPs in cancer cells is primarily attributed to lipid peroxidation rather than ROS production.

Ferroptosis is a type of programmed cell death characterized by the accumulation of lipid peroxides. It occurs when reactive oxygen species (ROS), such as hydroxyl radicals (•OH), extract electrons from lipid molecules in the cell membrane. This process, known as lipid peroxidation, leads to the disruption of membrane integrity and ultimately cell death. Unlike other forms of cell death, ferroptosis is specifically driven by the iron-dependent accumulation of lipid ROS, making it distinct and significant in various physiological and pathological contexts. In our study, Au@MIL NPs appear to induce lipid peroxidation through a mechanism that does not involve the production of traditional ROS intermediates. Instead, Au@MIL NPs directly extract electrons from lipid molecules. This suggests that Au@MIL NPs facilitate lipid oxidation and potentially ferroptosis through an alternative pathway that bypasses the generation of conventional ROS. The unique behavior of Au@MIL NPs in directly extracting electrons from lipids highlights its distinct mode of action, which could offer advantages in controlling oxidative stress and cell death in specific applications where ROS production is undesirable or needs to be minimized.

### Example 8 Examination of Stability for Au@MIL NPs

The hemolytic property of Au@MIL NPs in defibrinated sheep blood was evaluated to ensure their safe circulation in blood vessels. Au@PEG NPs served as a control reference. No hemolysis of erythrocytes was observed, indicating the feasibility and safety of administering Au@MIL NPs in mice. Next, the stability of Au@MIL NPs was examined. It was found that Au@MIL NPs can be safely stored in water before further in vitro and in vivo investigations. No apparent aggregation-induced precipitation or morphological changes were observed after incubation at 4° C. or 25° C. for 7 days, indicating satisfactory stability. It shows intact Au@MIL NPs at 4° C. and 25° C. before and after 7 days (red arrows indicating the presence of MILs). Furthermore, the anti-cancer efficacy of Au@MIL remained evident at 4° C. and 25° C. after 7 days of storage, demonstrating sustained performance and potential for further applications.

### Example 9 Tumor Regression in Orthotopic Xenograft Mice

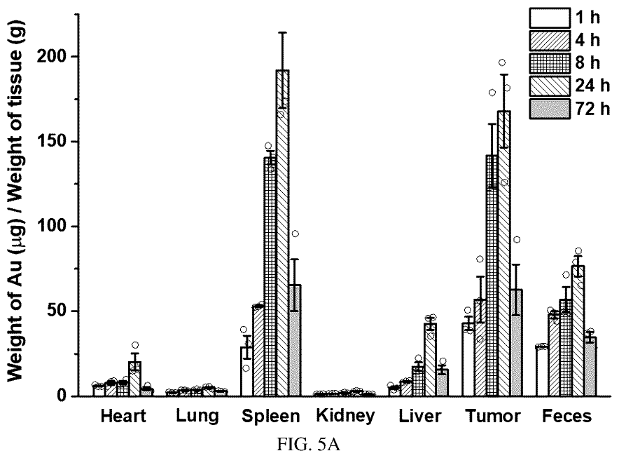

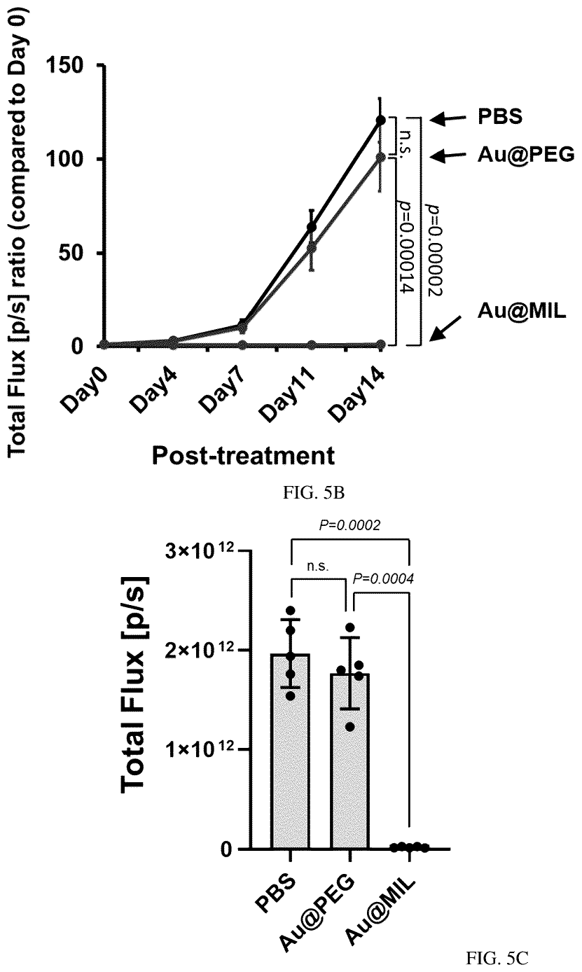

To demonstrate the efficacy of Au@MIL for in vivo anti-tumor, we prepared Hep G2-Red-FLuc orthotopic hepatocellular carcinoma (HCC) xenograft mice and administered a single dose of Au@MIL, Au@PEG, and sterilized PBS via intravenous injection. Biodistribution analysis reveals apparent accumulation of Au@MIL in the spleen, liver, and feces, indicating typical metabolic processes through hepatobiliary clearance (FIG. 5A). Notably, substantial NPs accumulation within the orthotopic HCC tumor site was observed in a time-dependent manner, peaking at 24 hours post-treatment, likely due to the enhanced permeability and retention effect. In anti-tumor experiments, the Au@MIL group showed significant tumor regression compared to PBS and Au@PEG treatments, as evidenced by an IVIS luminance signal ratio of less than 1 (compared to Day 0, with no therapy) over the 14-day post-treatment period (FIG. 5B). No significant changes in body weight were observed among the groups. After the therapeutic mice were sacrificed on Day 14 post-treatment, ex vivo IVIS detection and histological analysis confirmed the lowest luminance signal and the smallest tumor area of HCC in the Au@MIL group (FIG. 5C). As expected, we observed abundant expression of cell death and lipid peroxidation markers within the cytoplasm of Au@MIL-treated HCC cells in tumor sections. Importantly, a single dose of Au@MIL significantly prolonged the survival rate of Hep G2-Red-FLuc HCC xenograft mice, with no deaths observed up to Day 50 post-treatment (FIG. 5D). Finally, we validated the in vivo biosafety of Au@MIL in immunocompetent C57BL/6 mice, finding no impact on murine body weight, serum/urine biochemical indices, or histological features of normal organs. These data suggest that Au@MIL has potential as a therapeutic strategy for HCC, leveraging electron transfer from cancer cells to Au NPs to combat tumors.

### Example 10 Comprehensive Evaluation of Au@MIL NPs: Cellular Uptake, Electron Transfer, Oxidative Stress, Spectral Analysis, and Cytotoxicity Under Hypoxic Condition

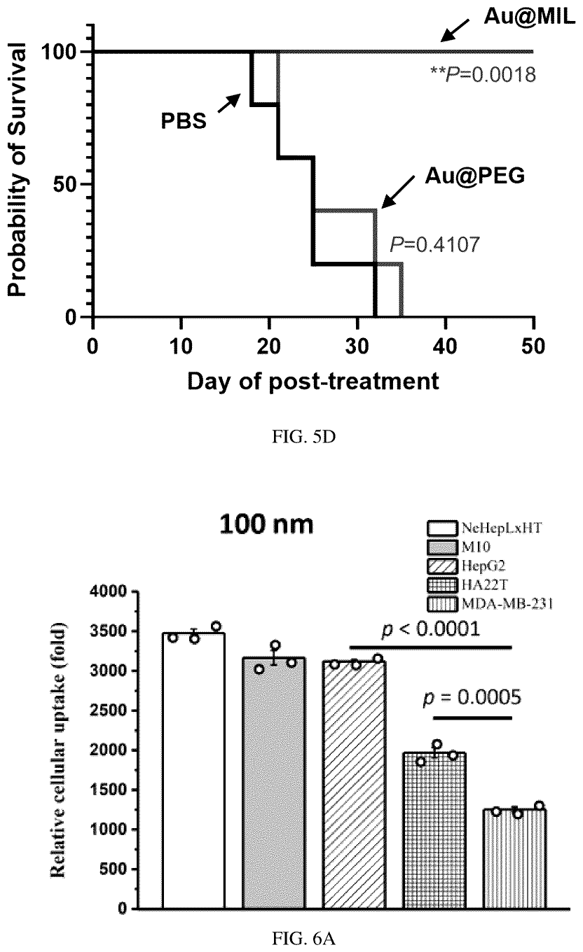

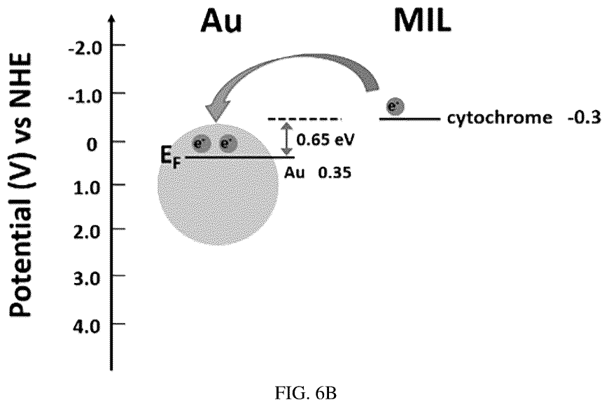

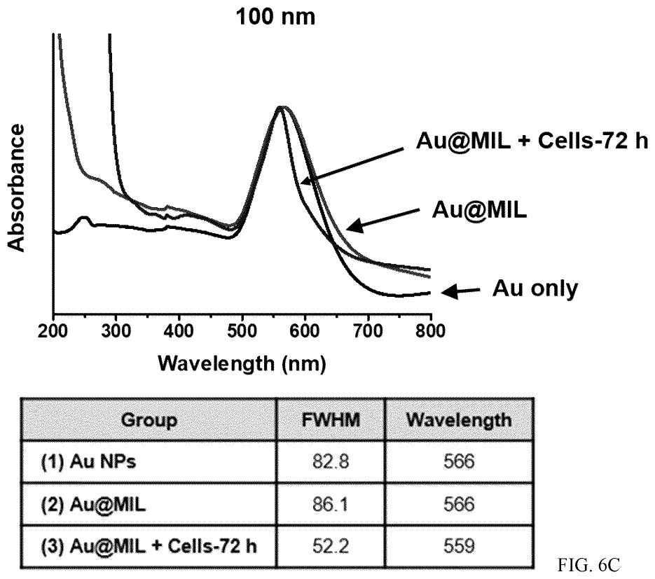

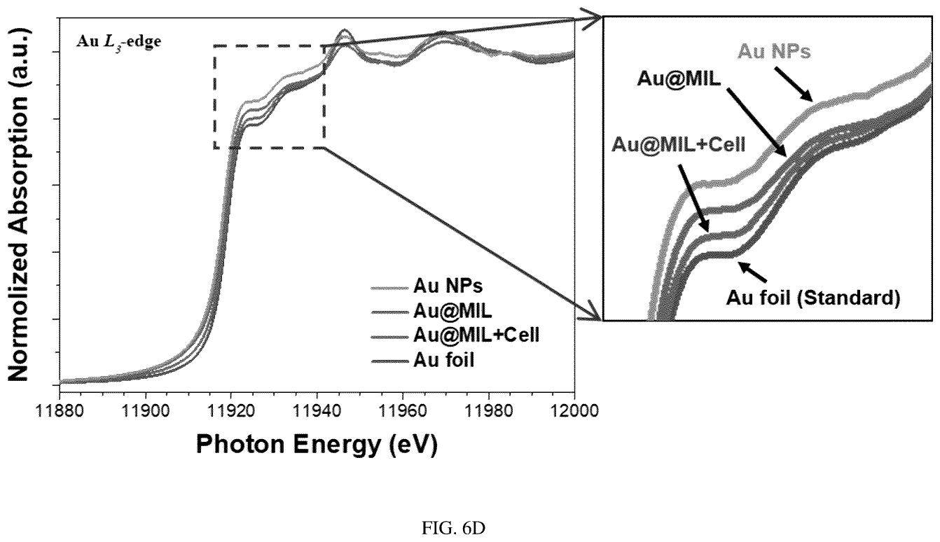

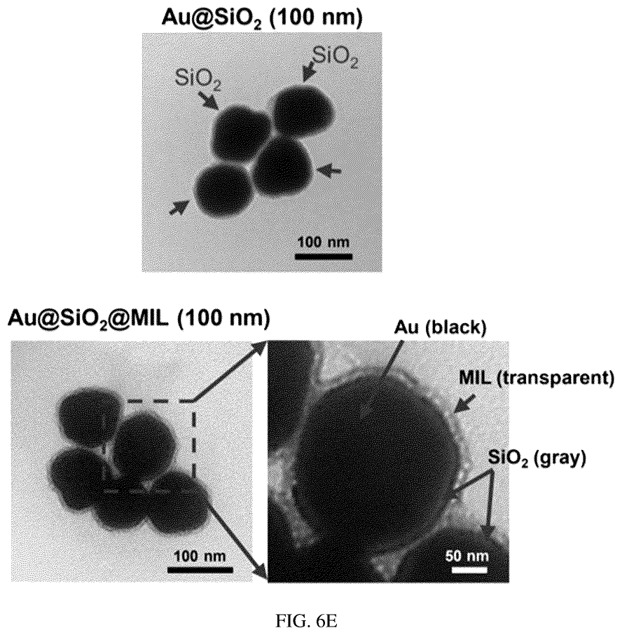

The presentation of cellular uptake results at 100 nm indicates a lack of clear trend regarding the efficacy of Au@MIL NPs in cancer cell eradication (FIG. 6A). While some cancer cells display higher uptake levels, others exhibit lower uptake. For example, Au@MIL NPs are taken up 2.5 times more by Hep G2 cells compared to MDA-MB- 231 cells and the colony assay survival rates are at 4% for Hep G2 cells and 15% for MDA-MB- 231 cells. In contrast, while the cellular uptake by HA22T cells is 1.6 times that of MDA-MB- 231 cells, the survival rates are at 32% for HA22T cells and 15% for MDA-MB- 231 cells. This discrepancy implies an inconsistency between the uptake of Au@MIL NPs and their efficacy in killing cancer cells.

The enhanced effectiveness of 100 nm Au NPs in killing cancer cells compared to 70 nm NPs may be attributed to their surface area and the number of cytochromes on their surface. While larger Au NPs have a smaller surface area per particle, their total surface area can be relatively large, especially at higher concentrations. Consequently, larger particles can accommodate more charge when combined with MIL. Additionally, the number of cytochromes in MIL coating the surface of 100 nm Au NPs is 3.4 times that of 70 nm particles, allowing for more efficient electron transfer. Cytochromes embedded in the outer membrane of Shewanella oneidensis MR-1 play the most crucial role in facilitating the extracellular electron transfer process. These factors likely result in more electrons being transferred to the larger 100 nm NPs, thereby more effectively disrupting the redox balance of cancer cells.

For the argument for the transfer of electrons from cancer cells to Au NPs through the membrane, cytochrome with a redox potential of −0.3 eV plays a crucial role. This potential is more negative than the Fermi level of gold (Au), ranging from +0.35 to +0.45 eV compared to the Normal Hydrogen Electrode (NHE) (FIG. 6B). Consequently, extracellular electrons can potentially flow to Au through cytochrome. Therefore, we conducted the following experiments to demonstrate that the death of cancer cells involves the electron transfer from cancer cells to Au NPs.

The oxidation-reduction process involves the transfer of electrons between molecules within cells and is necessary for maintaining cellular activities. However, when cells lack sufficient antioxidant capacity, it can lead to oxidative stress, which damages cellular structures and functions. To confirm that the oxidation-reduction process is impaired within cells, adding antioxidants can help balance this process, protecting cells from damage. Accordingly, we have found the inclusion of antioxidant vitamin E effectively inhibits the oxidative-reduction (redox) damage inflicted by 100 nm Au@MIL on cancer cells.

Additional evidence is the observed a notable phenomenon wherein there is blue shift and narrowing of the bandwidth of the Au surface plasmon band (SPR) for Au@MIL after incubation with cancer cells. The SPR of noble metallic NPs is highly sensitive to the charge density accumulated on the nanostructures. Theoretical calculations and experimental observation³¹⁻³³ suggest that the plasmonic frequency is directly correlated with the electron density within the NP. An increase in the concentration of free electrons leads to a blue shift in the plasmon resonance wavelength. Excess electrons on the surface of an Au NP can modify the collective oscillation of electrons (plasmons), impacting the absorption or scattering of light and causing the resonance wavelength to shift towards the blue end of the spectrum. Specifically, in the case of 100 nm Au, Au@MIL incubated with cells for 72 hours clearly displayed a blue shift to 559 nm in the SPR, whereas both Au and Au@MIL without cell incubation exhibited identical surface plasmon wavelength (566 nm) towards the red end of the spectrum (FIG. 6C). Additionally, we found that the bandwidth of the SPR is inversely proportional to the electron density³³. As electron concentration increases, the bandwidth decreases. Indeed, we observed the bandwidth narrow down from 86 nm to 52 nm in Au@MIL.

The XANES technique is very sensitive to small changes in Au 5d counts, especially the first spike at the beginning of the edge jump (also known as the white line). FIG. 6D shows the normalized Au L3-edge XANES of the three NPs (Au NPs, Au@MIL, and Au@MIL after cancer cells incubation) and Au metal (bulk). The intensity systematic of the resonance at the threshold (white line) associated with a 2p3/2 to 5d5/2; 3/2 dipole transition probing the unoccupied densities of d states at the Fermi level. In principle, the Au 5d orbital is fully filled, but due to the s-d hybridization, there will still be some vacancies, resulting in the intensity of the white line of Au metal. As Au is nanosized, the intensity of the white line will also increase.³⁴ When Au NPs are coated with an organic membrane, charge redistribution will also occur, thereby affecting the intensity of the white line. When Au@MIL interacts with cells, we observe a significant decrease in white line intensity, which means that the electron orbitals of Au 5d are filled with more electrons. In other words, more electrons are transferred from the cells to the Au NPs.

Finally, to directly demonstrate the electron flow into Au NPs, an insulating SiO₂ layer (6 nm) was introduced between Au NPs and MIL (FIG. 6E). The live and dead cells in Hep G2 cells were received a 48-h treatment with 70 nm and 100 nm Au@SiO₂@MIL at a concentration of 300 ppm Au. Nuclei, live cells, and dead cells were stained with Hoechst 33342, calcein AM, and EthD-1, respectively. Remarkably, the death of cancer cells no longer occurs with a SiO₂ insulator, clearly suggesting electron transfer from cancer cells to Au NPs.

The last but not least, in cancer treatment, it is known that tumor hypoxia can lead to increased resistance of cancer cells, reducing the effectiveness of treatments. Hypoxia also promotes the invasiveness and metastasis of cancer cells, further complicating treatment. Additionally, hypoxic conditions induce genetic and metabolic changes in tumor cells, making them more adaptable to harsh environments, thereby increasing the complexity and failure rate of treatments. Therefore, effective strategies targeting tumor hypoxia are crucial for improving the success rate of cancer therapies.

Thus, we tested cancer cells in hypoxic condition because the entire research hinges on the electron transfer from cancer cells to Au NPs, disrupting the cancer cell redox balance. As expected, the significant results show that Au@MIL still effectively caused damage to cancer cells under hypoxic condition. The fact that Au@MIL can still effectively target this imbalance under hypoxia underscores its robustness and specificity in targeting cancer cell vulnerabilities. However, it is worth mentioning that the overall lethality of cancer cells under hypoxic condition is not as high as under non-hypoxic conditions, with a lethality ratio of 1:2 based on live & dead observation. The lower lethality of hypoxia cancer cells when treated with Au@MIL NPs is likely due to the differences in their redox balance, metabolic adaptations, activation of survival pathways, and microenvironmental factors. These differences enable hypoxia cancer cells to better withstand the oxidative stress induced by the Au@MIL NPs, resulting in a lower death rate. A topic is worthy of further investigation.

1. A gold nanoparticle composition comprising:
a gold nanoparticle;
a liposome encapsulating the gold nanoparticle; and
a heme integrated into the liposome.

2. The gold nanoparticle composition of claim 1, wherein the gold nanoparticle ranges from 1 nm to 500 nm in diameter.

3. The gold nanoparticle composition of claim 1, wherein the gold nanoparticle is conductive.

4. The gold nanoparticle composition of claim 1, wherein a surface of the gold nanoparticle is modified.

5. The gold nanoparticle composition of claim 1, wherein a surface of the gold nanoparticle is modified with PEGylation.

6. The gold nanoparticle composition of claim 1, wherein the heme is contained in a cytochrome.

7. The gold nanoparticle composition of claim 6, wherein the cytochrome comprises ten hemes.

8. The gold nanoparticle composition of claim 6, wherein the cytochrome is MtrA, MtrB, MtrC, OmcA or OmcB.

9. The gold nanoparticle composition of claim 1, wherein the heme comprises Fe, and a molecular ratio of Fe to the heme ranges from 1:1 to 1:4.

10. The gold nanoparticle composition of claim 1, wherein a molecular ratio of the gold nanoparticle to the liposome ranges from 1 to 15.

11. The gold nanoparticle composition of claim 6, wherein a molecular ratio of the cytochrome to the gold nanoparticle ranges from 1 to 6000.

12. The gold nanoparticle composition of claim 1, wherein the liposome and the heme have a redox potential ranging from-0.4 eV to +0.3 eV.

13. The gold nanoparticle composition of claim 1, wherein the liposome and the heme are provided by Shewanella oneidensis MR-1.

14. A pharmaceutical composition comprising an effective amount of the gold nanoparticle composition of claim 1 and optionally a pharmaceutically acceptable carrier.

15. A method for treating cancer comprising administering the gold nanoparticle composition of claim 1 to a subject in need.

16. The method of claim 15, wherein the cancer is a solid cancer.

17. The method of claim 15, wherein the cancer is selected from the group consisting of squamous cell cancer, lung cancer, cancer of the peritoneum, hepatocellular cancer, gastric or stomach cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, head and neck cancer, lymphomas, leukemias, myelomas and myeloproliferative neoplasms.

18. The method of claim 15, wherein the cancer is selected from the group consisting of liver cancer, lung cancer, pancreatic cancer, breast cancer, cervical cancer, and colorectal cancer.

19. A method for inducing lipid oxidation, membrane potential change and/or reactive oxygen species (ROS) generation comprising administering the gold nanoparticle composition of claim 1 to a subject in need.

20. The method of claim 19, wherein the lipid oxidation or membrane potential change occurs in mitochondrial or endoplasmic reticulum.
